(19) United States
(12) Patent Application Publication
(10) Pub. No.: US 2020/0048691 A1
(43) Pub. Date: Feb. 13, 2020

(54) SWITCH-LIKE ISOTHERMAL DNA AMPLIFICATION DEMONSTRATING A NON-LINEAR AMPLIFICATION RATE

(71) Applicant: Montana State University, Bozeman, MT (US)

(72) Inventors: Stephanie E. McCalla, Bozeman, MT (US); Tomas Gedeon, Bozeman, MT (US)

(21) Appl. No.: 16/605,367

(22) PCT Filed: Apr. 17, 2018
(86) PCT No.: PCT/US2018/027918

§ 371 Date: Oct. 15, 2019

Related U.S. Application Data
(60) Provisional application No. 62/486,453, filed on Apr. 17, 2017.

(51) Int. Cl. C12Q 1/6844

(52) U.S. Cl. CPC C12Q 1/6844

(57) ABSTRACT

The presently-disclosed subject matter generally relates to methods, systems, compositions, and kits for the rapid, isothermal amplification of nucleic acids. The chemistry of the presently-disclosed amplification technique is isothermal, can be adapted to respond to a broad range of input target molecules, and results in a novel, biphasic reporter oligonucleotide amplification scheme with a high-gain second phase “burst” demonstrating a non-linear amplification rate (i.e., cooperative Hill kinetics). The switch-like amplification technique acts decisively to a true signal while filtering out noise, thus eliminating high levels of non-specific background amplification and false-positives.

### CROSS-REFERENCE TO RELATED APPLICATION

This application is a National Stage Application of PCT International Patent Application No. PCT/US2018/027918 filed on Apr. 17, 2018, under 35 U.S.C. § 371, which designates the United States and depends from and claims priority to U.S. Provisional Application No. 62/486,453 filed Apr. 17, 2017, the entire contents of which are incorporated herein by reference.

### GOVERNMENT RIGHTS

This invention was made with government support under UL1 TR002319 awarded by the National Center for Advancing Translational Sciences of the National Institutes of Health. The government has certain rights in the invention.

### REFERENCE TO SEQUENCE LISTING SUBMITTED ELECTRONICALLY

The instant application contains a Sequence Listing which has been submitted electronically in ASCII format and is hereby incorporated by reference in its entirety. Said ASCII copy, created on Apr. 16, 2017, is named “SEQENCE LISTING_ST25” and is 13 KB bytes in size.

### TECHNICAL FIELD

The presently-disclosed subject matter generally relates to methods, systems, compositions, and kits for the rapid, isothermal amplification of nucleic acids that acts decisively to a true signal while filtering out noise, thus eliminating high levels of non-specific background amplification and false-positives.

### BACKGROUND

Target nucleic acid detection by primer-mediated amplification has been widespread for many years, with several new nucleic acid amplification based detection and diagnosis approaches being developed and used in recent years. The most common amplification technique is the polymerase chain reaction (PCR), which remains an important detection method because of its reliability and specificity. PCR is a simple and flexible method for the reproduction and limited modification of the nucleotide sequence of a target nucleic acid. The PCR technique requires the cycling of temperatures to proceed through the steps of denaturation of the double-stranded DNA, annealing of short oligonucleotide primers, and extension of the primer along the template by a thermostable polymerase. While advances in the engineering have shortened these reaction times to 20-30 minutes, in order to achieve the amplification, a protocol which passes through at least two different temperature steps in 20 to 40 cycles must be executed. As such, the PCR technique requires specialized instrumentation and, a steep power requirement for the thermocycling units, and remains a relatively time-consuming process as only a doubling of the target nucleic acid is possible in each cycle.

Various isothermal amplification techniques have been developed to circumvent the need for temperature cycling and to decrease the time needed for detection. Isothermal oligonucleotide amplification chemistries have become increasingly popular due to their simplicity and adaptability to a variety of systems. For example, enzyme□free strand displacement amplification cascades can rapidly produce free oligonucleotides with nanomolar input trigger concentrations. Other oligonucleotide amplification reactions rely on polymerase to extend a template□bound oligonucleotide trigger and a nicking endonuclease to free the newly made product. The most common example of this reaction scheme is the exponential amplification reaction, or EXPAR, and the linear amplification modification thereof.

In the linear modification of EXPAR, a complementary oligonucleotide with a recognition sequence for a nicking endonuclease hybridizes onto a single-strand target nucleic acid. After the nicking, the oligonucleotide now consists of two shorter oligonucleotides which are bound onto the target nucleic acid. The experimental conditions, the length of the two cleaved oligonucleotides and the reaction temperature, are selected such that the shorter oligonucleotide, but not the longer one, dissociates from the target nucleic acid. The longer oligonucleotide, which has remained on the target nucleic acid, now serves as a primer, so that the single-strand region of the target nucleic acid is again filled in. In the next cycle, the nicking endonuclease again cleaves the single strand, and a further short oligonucleotide dissociates from the target nucleic acid. The detection of the target nucleic acid can be effected by mass spectrometry detection the short oligonucleotides formed in this reaction.

In the exponential modification of EXPAR, the dissociating oligonucleotide of the linear amplification is used in order to form a new primer which can bind to a so-called amplification template. This amplification template is added to the reaction mixture in addition to the target nucleic acid. The amplification template possesses a recognition site for the nicking endonuclease. In addition, the dissociating oligonucleotide can bind both at the 5′ end also at the 3′ end. In the first step, the oligonucleotide dissociated in the previous cycle binds to the complementary sequence which lies 5′ from the recognition site for the nicking endonuclease, forming a transient complex between the dissociated oligonucleotide and the amplification template. In the second step, the oligonucleotide, which now serves as a primer, is elongated at its 3′ end over the whole amplification template. A double-strand amplification template is thus formed which possesses a recognition site for the nicking endonuclease, and after cleavage by the nicking endonuclease, once again releases a short dissociating oligonucleotide which once again can bind to a further amplification template. In this method also, the detection of the dissociated short oligonucleotides can also be effected by means of mass spectrometry.

However, while EXPAR has several advantages over PCR, EXPAR can result in high levels of non-specific background amplification and can generate false-positives.

Switch□like responses to input stimuli are ubiquitous in nature. This switching behavior is common in cell signaling, transcription, and genetic regulatory networks; it is commonly accepted that these switches react decisively to a true signal while filtering out noise. Several studies have reported switch□like behavior in synthetic biochemical systems. Ion channels can be repurposed into biosensor switches by preventing channel dimerization in the presence of a target antigen, thus turning on in the presence of target. DNA oscillators can switch between an “on” and “off” state by combining DNA degradation with a DNA amplification reaction. It was noted that this oscillatory effect could be achieved through non□linear DNA amplification instead of non□linear DNA degradation, but the former was difficult to obtain and manipulate and was therefore not an option when creating a DNA circuit. Structure□switching sensors such as aptamers and molecular beacons change conformation in the presence of a specific target molecule. When properly designed, structure□switching biosensors can also create ultrasensitive cooperative Hill kinetics: biosensors with two cooperative binding sites produce an ultrasensitive response if the affinity of the target for the second site is altered by target association to the first site. These exciting biomimetic systems typically produce outputs with nanomolar trigger inputs. A single cell can contain as few as 10 microRNA molecules per cell, and clinically relevant DNA and RNA concentrations range from hundreds of picomolars to attomolar in range. Clinically relevant protein concentrations are often in the femtomolar range. While previous studies explored sensors that are controlled switches, most do not have the subsequent high□gain amplification required for low target concentrations.

Thus, while isothermal oligonucleotide amplification reactions, such as EXPAR, are broadly used across a variety of disciplines (such as DNA circuits and logic gates, miRNA detection, aptamer□based analyte detection, RNA detection, and genomic DNA detection) there is a need in the art for novel isothermal oligonucleotide reactions that overcome the limitations of the prior art. More particularly, there remains a need for a switch-like amplification reaction that acts decisively to a true signal while filtering out noise, thus eliminating high levels of non-specific background amplification and false-positives.

### SUMMARY

Accordingly, the presently disclosed subject matter relates to rapid, isothermal, and biphasic nucleic acid amplification reaction scheme with an endogenous switching mechanism. The instantly-disclosed amplification technique exploits a naturally occurring stall in the amplification reaction, which produces a low□level signal. Upon surpassing a threshold, the reaction enters a high gain second phase “burst” demonstrating a non-linear amplification rate (e.g., cooperative Hill kinetics), producing an oligonucleotide concentration that ranges from ten to one hundred times the first phase plateau. The amplification technique acts decisively to a true signal while filtering out noise, thus eliminating high levels of non-specific background amplification and false-positives. Further, the high□gain “burst” demonstrating a non-linear amplification rate (e.g., cooperative Hill kinetics) allows for the instantly-disclosed isothermal amplification technique to detect very low levels of target nucleic acid molecules (e.g. but not limited to, ≤10 picomolar, ≤1 picomolar, or even the femtomolar range (e.g., ≤100 femtomolar, ≤10 femtomolar, ≤1 femtomolar)). Output kinetics can be tuned to control reaction acceleration in the second phase, resembling definitive switch turn□on. Additionally, reaction design using controlled DNA association thermodynamics give some control over first phase kinetics. Proteins, genomic bacterial DNA, viral DNA, microRNA, or mRNA can be transduced into many oligonucleotide triggers, making this technique applicable to a broad range of biological sensors and target molecules.

Some embodiments of the presently-disclosed subject matter provide methods of detecting a target oligonucleotide sequence (X). In some embodiments, the methods includes forming a reaction mixture that comprises: (1) a target nucleic acid comprising a target oligonucleotide sequence (X); (2) a first antisense template (X′R1t′Yp); and (3) a second antisense template (t′YpR2t′Yp). In some embodiments, the first antisense template (X′R1t′Yp) comprises from 3′ to 5′: (a) a first sequence of nucleotides (X′) that is at least substantially complementary to the target oligonucleotide sequence (X); (b) a second sequence of nucleotides (R1) of an anti-sense strand of a first nicking enzyme binding site; and (c) a third sequence of nucleotides (t′Yp) that is at least substantially complementary to a reporter oligonucleotide sequence (tYp). In some embodiments, the third sequence of nucleotides (t′Yp) comprises from 3′ to 5′: (i) a toehold nucleotide sequence (t′); and (ii) a palindromic nucleotide sequence (Yp). In some embodiments, the third sequence of nucleotides (t′Yp) is non-complementary to the target oligonucleotide sequence (X). In some embodiments, the second antisense template (t′YpR2t′Yp) comprises from 3′ to 5′: (a) a fourth sequence of nucleotides comprising t′Yp; (b) a fifth sequence of nucleotides (R2) of an anti-sense strand of a second nicking enzyme binding site; and (c) a sixth sequence of nucleotides comprising t′Yp. In some embodiments, the two palindromic nucleotide sequences (Yp) of the second antisense template (t′YpR2t′Yp) cause the second antisense template (t′YpR2t′Yp) to form a palindrome and fold into a stem and loop configuration. The reaction mixture further includes a polymerase, a first nicking enzyme that nicks at the first nicking enzyme binding site, a second nicking enzyme that nicks at the second nicking enzyme binding site, and nucleotides. The methods further include subjecting the reaction mixture to essentially isothermal conditions at a reaction temperature to amplify the reporter oligonucleotide sequence (tYp) at a non-linear amplification rate, and detecting the reporter oligonucleotide sequence (tYp).

In some aspects of the methods of detecting a target oligonucleotide sequence (X), the non-linear amplification rate of the reporter oligonucleotide sequence (tYp) demonstrates cooperative Hill kinetics.

In some aspects of the methods of detecting a target oligonucleotide sequence (X), the amplification of the reporter oligonucleotide sequence (tYp) is biphasic. In some aspects, the first phase linearly amplifies the oligonucleotide sequence (tYp) and the second phase amplifies the reporter oligonucleotide sequence (tYp) at a non-linear amplification rate.

In some aspects of the methods of detecting a target oligonucleotide sequence (X), said method can detect the target oligonucleotide sequence (X) at a concentration of ≤10 picomolar.

In some aspects of the methods of detecting a target oligonucleotide sequence (X), the reporter oligonucleotide sequence (tYp) is linearly amplified from the steps including: (A) forming a duplex (D1) comprising the target oligonucleotide sequence (X) and the first antisense template (X′R1t′Yp); (B) extending, using the polymerase, the target oligonucleotide sequence (X) of the duplex (D1) along the first antisense template (X′Rlt′Yp) to form an extended target oligonucleotide sequence comprising a sense sequence complementary to the first antisense template (X′R1t′Yp); (C) nicking, with the first nicking enzyme, at the first nicking enzyme binding site on the sense strand of the duplex (D1) to produce the reporter oligonucleotide sequence (tYp); and (D) repeating steps (B) and (C) to thereby linearly amplify the reporter oligonucleotide sequence (tYp).

In some aspects of the methods of detecting a target oligonucleotide sequence (X), the reporter oligonucleotide (tYp) is non-linearly amplified from the steps including: (A) forming a duplex (D2) comprising the reporter oligonucleotide sequence (tYp) and the second antisense template (t′YpR2t′Yp), wherein binding of the reporter oligonucleotide sequence (tYp) to the toehold site (t′) unfolds the stem and loop configuration of the second antisense template (t′YpR2t′Yp); (B) extending, using the polymerase, the reporter oligonucleotide sequence (tYp) of the duplex (D2) along the second antisense template (t′YpR2t′Yp) to form an extended reporter oligonucleotide sequence comprising a sense sequence complementary to the second antisense template (t′YpR2t′Yp); (C) nicking, with the second nicking enzyme, at the second nicking enzyme binding site on the sense strand of the duplex (D2) to produce the reporter oligonucleotide sequence (tYp); and (D) repeating steps (B) and (C) to thereby non-linearly amplify the reporter oligonucleotide sequence (tYp). In aspects, the non-linear amplification rate of the reporter oligonucleotide sequence (tYp) demonstrates cooperative Hill kinetics

In some aspects of the methods of detecting a target oligonucleotide sequence (X), the first nicking binding site and the second nicking binding site are identical.

In some aspects of the methods of detecting a target oligonucleotide sequence (X), the first nicking site and the second nicking site are nicked by the same nicking enzyme.

In some aspects of the methods of a detecting a target oligonucleotide sequence (X), the first sequence of nucleotides (X′) is completely complementary to the target oligonucleotide sequence (X).

In some aspects of the methods of a detecting a target oligonucleotide sequence (X), the third sequence of nucleotides (t′Yp) is completely complementary to the reporter oligonucleotide sequence (tYp).

In some aspects of the methods of a detecting a target oligonucleotide sequence (X), the 3′ terminus of the first antisense template (X′R1t′YP) and the 3′ terminus of the second antisense template (t′YpR2t′YP) are blocked.

In some aspects of the methods of a detecting a target oligonucleotide sequence (X), detecting the reporter oligonucleotide sequence (tYp) is performed at least partially by luminescence spectroscopy or spectrometry, fluorescence, fluorescence spectroscopy or spectrometry, mass spectrometry, liquid chromatography, fluorescence polarization, colorimetry, and/or electrophoresis.

In some aspects of the methods of a detecting a target oligonucleotide sequence (X), detecting reporter oligonucleotide sequence (tYp) comprises detecting amplification of the reporter oligonucleotide sequence (tYp). In some aspects, the step of detecting amplification of the reporter oligonucleotide sequence (tYp) is performed at least partially by a technique selected from the group consisting of luminescence spectroscopy or spectrometry, fluorescence, fluorescence spectroscopy or spectrometry, mass spectrometry, liquid chromatography, fluorescence polarization, colorimetry, and electrophoresis.

In some aspects of the methods of detecting a target oligonucleotide sequence (X), detecting reporter oligonucleotide sequence (tYp) comprises detecting an amplification rate of the reporter oligonucleotide sequence (tYp). In some aspects of the methods of detecting a target oligonucleotide sequence (X), the step of detecting the amplification rate of the reporter oligonucleotide sequence (tYp) is performed at least partially by luminescence spectroscopy or spectrometry, fluorescence, fluorescence spectroscopy or spectrometry, mass spectrometry, liquid chromatography, fluorescence polarization, colorimetry, and/or electrophoresis.

In some aspects of the methods of detecting a target oligonucleotide sequence (X), the target nucleic acid comprising a target oligonucleotide sequence (X) is obtained from a sample derived from an animal. In some aspects, the sample is blood, serum, mucus, saliva, urine, or feces.

In some aspects of the methods of detecting a target oligonucleotide sequence (X), the target nucleic acid comprising a target oligonucleotide sequence (X) is any synthetic or natural RNA molecule, including mRNA, microRNA, and siRNA. In other aspects, the target nucleic acid comprising a target oligonucleotide sequence (X) is any synthetic or natural DNA molecule, including genomic DNA, mitochondrial DNA, cDNA derived from reverse transcription of mRNA, microRNA, or siRNA, and said method comprises a step of denaturing said target nucleic acid comprising a target oligonucleotide sequence (X) prior to forming the reaction mixture.

In some aspects of the methods of detecting a target oligonucleotide sequence (X), the polymerase is a warm start polymerase.

In some aspects of the methods of detecting a target oligonucleotide sequence (X), the amplification of the reporter oligonucleotide sequence (tYp) is performed at about 55° C. to about 60° C.

In some aspects of the methods of detecting a target oligonucleotide sequence (X), the reporter oligonucleotide sequence (tYp) is from 8-30 nucleotides in length. In some aspects of the methods of detecting a target oligonucleotide sequence (X), the toehold site (t′) of the first, third, fourth, and fifth sequence of nucleotides is from 3-8 nucleotides in length. In some aspects of the methods of detecting a target oligonucleotide sequence (X), the palindrome of the second antisense template (t′YpR2t′Yp) is from 4-22 nucleotides in length.

In some aspects of the methods of detecting a target oligonucleotide sequence (X), the palindrome of the second antisense template (t′YpR2t′Yp) has a melting temperature that is greater than the reaction temperature, but less than 90° C.

In some aspects of the methods of detecting a target oligonucleotide sequence (X), the duplex (D2) has a melting temperature that is less than the reaction temperature plus 5° C.

In some aspects of the methods of detecting a target oligonucleotide sequence (X), the nicking enzyme is selected from the group consisting of Nt.BstNBI, Nt.BspQI, Nb.BBvCl, Nb.Bsml, Nb.BsrDI, Nb.BstI, Nt.AlwI, Nt.BbvCI, Nt.CviPII, Nt.BsmAI, Nb.Bpu1oI, and Nt.Bpu10I.

These and additional embodiments and features of the presently-disclosed subject matter will be clarified by reference to the figures and detailed description set forth herein.

It is understood that both the preceding summary and the following detailed description are exemplary and are intended to provide further explanation of the disclosure as claimed. Neither the summary nor the description that follows is intended to define or limit the scope of the disclosure to the particular features mentioned in the summary or description.

### BRIEF DESCRIPTION OF THE DRAWINGS

FIGS. 1A-B depict schematic diagrams illustrating an embodiment of the reaction scheme of instantly-disclosed rapid, isothermal, biphasic nucleic acid amplification technique having an endogenous switching mechanism and having a high□gain second phase “burst” demonstrating hill-type kinetics. FIG. 1A depicts a schematic diagram illustrating the reaction scheme of the linear phase of the biphasic amplification of a reporter oligonucleotide sequence (tYp). FIG. 1B depicts a representative reaction scheme of the non-linear amplification of a reporter oligonucleotide sequence (tYp), the non-linear amplification demonstrating hill-type kinetics.

FIGS. 2A-B depict potential pathways of the biphasic nucleic acid amplification. FIG. 2A depicts a schematic diagram of potential pathways of the biphasic nucleic acid amplification, particularly amplification of a reporter oligonucleotide sequence (tYp). FIG. 2B depicts a representative reaction trace of template (t′YpR2t′YP), particularly template LS2. The reaction stages are labeled with the proposed reaction mechanisms of FIG. 2A that govern each reaction stage.

FIGS. 3A-E depict representative biphasic amplification reaction output. FIG. 3A depicts representative amplification traces that demonstrate that nucleic acid amplification (reporter oligonucleotide sequence (tYp)) using various templates (t′YpR2t′Yp) is correlated to fluorescence, which increases and plateaus at approximately the same level as previously reported optimized EXPAR reactions (dotted lines). After a lag period, the nucleic acid (reporter oligonucleotide sequence (tYp)) output jumps into a high gain “ON” region. Template (t′YpR2t′Yp) names are labelled next to corresponding output traces; template sequences can be found in Table 1. Biphasic DNA amplification output is shown in solid lines. FIGS. 3B-E depict the reaction tube images of LS2 and EXPAR1 amplification templates were captured under fluorescent light using an LED transilluminator (470 nm excitation) and an iPhone SE. FIG. 3B depicts LS2 template, 60 minutes; FIG. 3C depicts EXPAR1 template, 15 minutes; FIG. 3D depicts LS2, 0 minutes; and FIG. 3E depicts EXPAR1, 0 minutes.

FIGS. 4A-F are amplification traces and graphs of corresponding inflection points that depict the correlation between amplification initiation and reporter oligonucleotide sequence (tYp) concentration. The real-time reaction output for three representative templates shows the dependence of the reaction on initial reporter oligonucleotide sequence (tYp) concentration, with fluorescence correlated to the produced reporter oligonucleotide sequence (tYp). Initial reporter oligonucleotide sequence (tYp) concentrations were increased tenfold between 100 fM and 10 μM unless otherwise indicated; darker color indicates higher initial reporter oligonucleotide sequence (tYp) concentrations. Horizontal lines in FIGS. 4D-F show inflection points when no trigger is added. FIG. 4A depicts that the dilution series of a standard EXPAR template (EXPAR1), which do not enter the second phase, even at high concentrations of trigger (10 μM). FIG. 4B depicts dilution series of the representative Type I template LS2 lowtG, which includes an extra trace at 20 μM initial reporter oligonucleotide sequence (tYp). FIG. 4C depicts the dilution series of the representative Type II template LS3. Calculated inflection points are shown for EXPAR1 (FIG. 4D), LS2 lowtG (FIG. 4E), and LS3 (FIG. 4F). For FIGS. 4D-E, dashed lines show fits; grey symbols show first inflection points and black symbols show the second inflection point. Error bars represent standard deviation of experimental triplicates.

FIG. 5 is a graph that demonstrates that weakening the loop structure of templates can result in slower reaction kinetics. Long random sequences (lrs) were added to four base looped templates after the nickase recognition site, resulting in a weaker template loop that produces the same product. The relative first inflection point is the average first inflection point divided by the average first inflection point of the base template without lrs; a value greater than one therefore signifies reduced first phase reaction kinetics. While a weakened loop has a modest e□ect on Type I templates (trigger Tm<reaction temperature+5° C., so Tm<60° C. at a reaction temperature of 55° C.), Type II templates (trigger Tm>reaction temperature+5° C., so Tm>60° C. at a reaction temperature of 55° C.) with weakened loops are much slower than their base template. Error bars represent standard deviations from at least three independent experiments, which all contained experimental replicates. *p<0.05, **p<0.01, ***p<0.001, Holm-Bonferroni t-test.

FIGS. 6A-B are charts that demonstrate the acceleration of trigger production in the second phase vs. DNA association thermodynamics. Type I and type II templates show two distinct behaviors in the second phase. FIG. 6A demonstrates type I templates have triggers that can dynamically dissociate from the template at the reaction temperature (trigger Tm<reaction temperature+5° C., so Tm<60° C. at a reaction temperature of 55° C.). If the cooperativity of trigger binding to the two open toeholds contributed to reaction acceleration in the second phase, then the thermodynamic di□erence between the first and second trigger binding events (ΔG5′toehold+ΔG3′toehold+ΔGpalindrome−ΔGloop)−ΔGtrigger:template would correlate with accelerating kinetics in the second phase. This is true of type I templates (Spearman's Rho=0.9667, p<1.7×10⁻⁴), but not type II templates (Spearman's Rho=0.6437, p<0.10). FIG. 6B demonstrates that type II templates have triggers that are stable at the reaction temperature (trigger Tm>reaction temperature+5° C., so Tm>60° C. at a reaction temperature of 55° C.), making loop closure and long trigger removal more di□cult. Long trigger removal, as described in FIG. 2A, is approximated by ΔGlong trigger:trigger+ΔGloop−ΔGlong trigger:template. This correlates with second phase acceleration of type II templates (Spearman's Rho=−0.9762, p<4.0×10⁻⁴), but not type I templates (Spearman's Rho=−0.3333, p<0.39). Inset of FIG. 6B is rescaled to show a zoomed in graph of type II template second phase acceleration.

FIGS. 7A-B depict the amplification traces and graphs of corresponding inflection points for the reactions using the mature miRNA miR-let7f-5p (5′-UGAGGUAGUAGUUGUAUAGUU-3′, SEQ ID NO: 73). miRNA miR-let7f-5p was transduced to trigger 5′-CCAAACTCCGGA-3′ (SEQ ID NO: 40, Table 1) in the reaction mixture by using either Transduction template LS31pG3let7f5pLNA (5′-TCCGGAGTTTGGTAATGACTCTAACTA+TACAATC+TACTACC+TC-3′ (PO₃) (SEQ ID NO: 74) or Transduction template LS3lowpG3let7f5p (5′-TCCGGAGTTTGGTAATGACTCTAACTATACAATCTACTACCTCA-3′ N_{H2}) (SEQ ID NO: 75), which were further used in combination with the DNA template LS3 lowpG3 (Table 1). This triggered the biphasic reaction chemistry demonstrating a non-linear amplification rate, and more specifically cooperative Hill kinetics.

FIGS. 8A-B depict the amplification traces and graphs of corresponding inflection points for the reactions using the he mature miRNA hsa-miR-223-3p (5′-UGUCAGUUUGUCAAAUACCCCA-3′, SEQ ID NO 76) was transduced to the trigger 5′-ATTCTCCGGA-3′ (SEQ ID NO: 29, Table 1) in the reaction mixture by using Transduction template LS2 (Table 1), which were further used in combination with the DNA template LS2 (Table 1). This triggered the biphasic reaction chemistry demonstrating a non-linear amplification rate, and more specifically cooperative Hill kinetics. Error bars were calculated from experimental triplicates.

FIG. 9 depicts template designs that can be used to create and “AND” logic gate. FIG. 9 depicts a splitting the reporter molecule using a single transduction template so that two reporters, t₁′Yp₁ and Yp₂t₂′, are produced, as well as generating two reporter molecules using two transduction templates so that two reporters, t₁′Yp₁ and Yp₂t₂′, are produced.

FIGS. 10A-C depict amplification switch designs. FIG. 10A depicts an inhibition switch design using an exonuclease that is specific for single-stranded DNA. FIG. 10B depicts a competition switch design. FIG. 10C depicts a relative amplification switch design.

### DETAILED DESCRIPTION

The following description of particular aspect(s) is merely exemplary in nature and is in no way intended to limit the scope of the invention, its application, or uses, which may, of course, vary. The invention is described with relation to the non-limiting definitions and terminology included herein. These definitions and terminology are not designed to function as a limitation on the scope or practice of the invention but are presented for illustrative and descriptive purposes only. While the processes and compositions are described as using specific a specific order of individual steps or specific materials, it is appreciated that steps or materials may be interchangeable such that the description of the invention may include multiple steps or parts arranged in many ways as is readily appreciated by one of skill in the art.

The presently-disclosed data demonstrates a rapid, biphasic nucleic acid amplification technique with an endogenous switching mechanism. This novel biphasic nucleic acid amplification technique is a simple, one-step isothermal amplification reaction, and therefore does not require temperature cycling. As such, the reaction requires less energy, hardware, and time as compared to techniques that require such temperature cycling, such as PCR. The instantly-disclosed amplification technique exploits a naturally occurring stall in the amplification reaction, which produces a low□level signal. Upon surpassing a threshold, the reaction enters a high□gain second phase “burst” and amplifies the reporter oligonucleotide sequence (tYp) at a non-linear amplification rate, producing a reporter oligonucleotide concentration that ranges from ten to one hundred times or more of the first phase plateau. In aspects, the non-linear amplification rate demonstrates cooperative Hill kinetics.

The presently-disclosed data demonstrates that this amplification technique acts decisively to a true signal while filtering out noise, thus eliminating high levels of non-specific background amplification and false-positives. Output kinetics can be tuned to control reaction acceleration in the second phase, resembling definitive switch turn□on. Additionally, reaction design using controlled DNA association thermodynamics give some control over first phase kinetics. A wide variety of target oligonucleotide sequences contained within a target nucleic acid, including those generated from or contained within proteins, genomic bacterial DNA, viral DNA, microRNA, or mRNA, can be transduced into many oligonucleotide triggers, making this technique applicable to a broad range of biological sensors and target molecules. The biphasic nature of this amplification technique demonstrating a definitive response and high-gain output makes it well suited for biomarker detection assays, particularly, for the recognition of low-concentration molecules in biological samples, as well as for DNA logic gates and other molecular recognition systems. Importantly, due to the high□gain “burst” in which the reporter oligonucleotide sequence (tYp) is amplified at a non-linear amplification rate (e.g., demonstrating cooperative Hill kinetics), the instantly-disclosed isothermal amplification technique can detect very low levels (picomolar or lower levels) of target nucleic acid molecules.

Accordingly, reference will now be made in detail to various embodiments of the instantly-disclosed methods/assays, systems, compositions, and kits for the rapid, isothermal amplification of nucleic acids.

The terminology used herein is for describing particular embodiments/aspects only and is not intended to be limiting. As used herein, the singular forms “a,” “an,” and “the” are intended to include the plural forms, including “at least one,” unless the content clearly indicates otherwise. “Or” means “and/or.” As used herein, the term “and/or” includes any and all combinations of one or more of the associated listed items. It will be further understood that the terms “comprises” and/or “comprising,” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof. The term “or a combination thereof” means a combination including at least one of the foregoing elements.

Unless otherwise defined, all terms (including technical and scientific terms) used herein have the same meaning as commonly understood by one of ordinary skill in the art to which this disclosure belongs. It will be further understood that terms such as those defined in commonly used dictionaries, should be interpreted as having a meaning that is consistent with their meaning in the context of the relevant art and the present disclosure, and will not be interpreted in an idealized or overly formal sense unless expressly so defined herein.

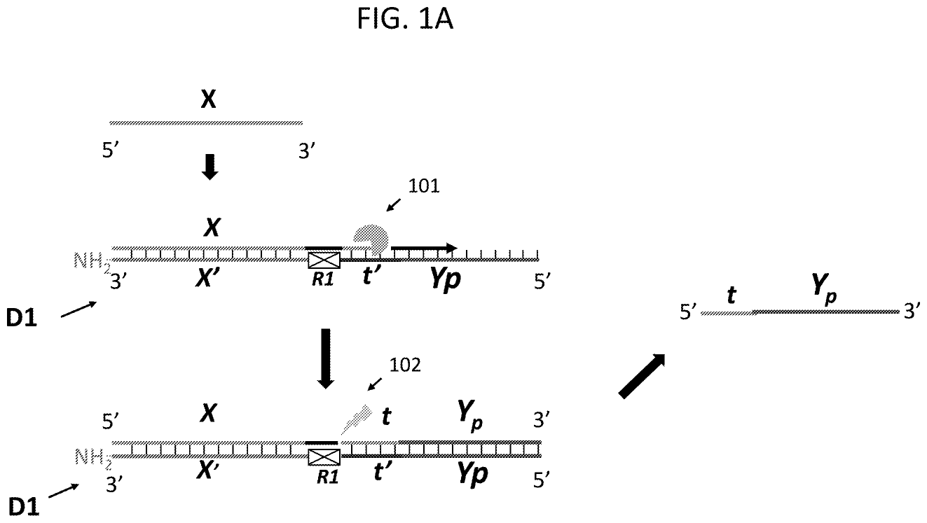

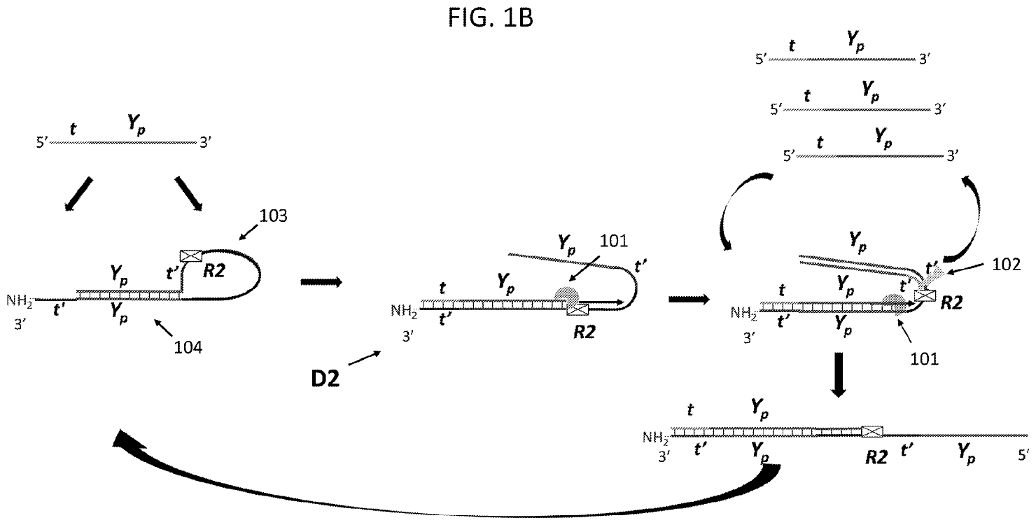

The novel, chemistry of the instantly-disclosed isothermal, biphasic nucleic acid amplification technique having an endogenous switching mechanism and a high□gain second phase “burst” demonstrating a non-linear amplification rate, such as cooperative Hill kinetics, is disclosed in FIGS. 1A-B. FIG. 1A depicts the reaction scheme of the linear phase of the biphasic amplification technique. In this first linear amplification phase, a target oligonucleotide sequence (X) is transduced into a reporter oligonucleotide sequence (tYp) (which may also be referred to herein as the “trigger”) by the anti-sense template (X′R1t′Yp) (which may be referred to herein as the “transduction template”). In embodiments, an antisense transduction template (X′R1t′Yp), depicted here as the antisense sequence (3′ to 5′), comprises a sequence (X′), that is at least substantially complementary to the target oligonucleotide sequence (X), and a sequence that is not substantially complementary to the target oligonucleotide and therefore does not hybridize with the target oligonucleotide during the reaction. The antisense sequence which is not substantially complementary to the target oligonucleotide comprises an anti-sense strand of a nicking enzyme binding site (R1) for a nicking enzyme (102); a toehold site (t′), and a palindromic sequence (Yp). The target oligonucleotide sequence (X) binds to the sequence (X′) of the antisense template (X′R1t′Yp), forming a duplex (D1). The target oligonucleotide sequence provides a 3′ hydroxyl group for an initial oligonucleotide extension. Using polymerase (101) and free nucleotides contained in the reaction mixture, the polymerase (101) extends the target oligo nucleotide sequence along the template (X′R1t′Yp) to create the sense strand comprising (XR1tYp). This extension creates a nicking enzyme recognition site (R1) on the sense strand of the now extended template. The nicking enzyme (102), that is also included in the reaction mixture, nicks the sense strand of the duplex (D1) at its nicking site, creating reporter oligonucleotide sequence (tYp). Once the reporter oligonucleotide sequence (tYp) is cleaved from the duplex (D1), the process of polymerase extension and nicking is repeated, linearly amplifying reporter oligonucleotide sequence (tYp).

FIG. 1B depicts a representative reaction scheme in which the reporter oligonucleotide sequence (tYp) is amplified at a non-linear amplification rate. In aspects, the non-linear amplification rate demonstrates cooperative Hill kinetics. The reporter oligonucleotide sequence (tYp) is amplified by the anti-sense template (t′YpR2t′Yp), which may be referred to herein as the “DNA template.” In embodiments, the anti-sense DNA template (t′YpR2t′Yp), depicted here as the antisense sequence (3′ to 5′), comprises two copies of a complementary sequence to the reporter oligonucleotide sequence joined by an anti-sense strand of a nicking enzyme recognition site. Thus, in some embodiments, the anti-sense DNA template (t′YpR2t′Yp), depicted here as the antisense sequence (3′ to 5′), comprises the toehold site (t′), the palindromic sequence (Yp), an anti-sense strand of a nicking enzyme binding site (R2) for a nicking enzyme (102), toehold site (t′), and palindromic sequence (Yp). The two palindromic nucleotide sequences (Yp) of the antisense DNA template (t′YpR2t′Yp) bind (forming a palindrome (104)) and cause the antisense DNA template (t′YpR2t′Yp) to form a palindrome and fold into a stem and loop configuration (103). The reporter oligonucleotide sequence (tYp), which is initially created from first phase of the bisphasic reaction as shown in FIG. 1A, binds to the sequence (t′Yp) of the antisense DNA template (t′YpR2t′Yp), forming a duplex (D2). Binding of the reporter oligonucleotide sequence (tYp) to either toehold site (t′) unfolds the stem and loop configuration of the antisense DNA template (t′YpR2t′Yp). Binding of the reporter oligonucleotide sequence (tYp) to the 3′ end of the antisense template (t′YpR2t′Yp) provide a 3′ hydroxyl group for an initial oligonucleotide extension. Using polymerase (101) and free nucleotides contained in the reaction mixture, the polymerase (101) extends the reporter oligonucleotide sequence (tYp) of the duplex (D2) along the antisense DNA template (t′YpR2t′Yp) to create the sense strand comprising (tYpR2tYp). This extension creates a nicking enzyme binding site (R2) on the sense strand of the now extended template. The nicking enzyme (102), that is also included in the reaction mixture, nicks the sense strand of the duplex (D1), creating reporter oligonucleotide sequence (tYp). Once the reporter oligonucleotide sequence (tYp) is cleaved from the duplex (D2), the process of polymerase extension and nicking is repeated, amplify the reporter oligonucleotide sequence (tYp) at a non-linear amplification rate. In aspects, the non-linear amplification rate demonstrates cooperative Hill kinetics.

The instantly-disclosed biphasic nucleic acid amplification technique includes many of the same basic components as the exponential amplification reaction for oligonucleotides (EXPAR). Both EXPAR and the biphasic DNA amplification reaction disclosed herein amplify a trigger sequence at a single reaction temperature (e.g., but not limited, to 55° C.) through the action of a thermophilic polymerase and a nicking enzyme. The main difference between the original EXPAR reaction and the instantly-disclosed biphasic target oligonucleotide amplification reaction is the palindromic sequence within the DNA template that causes this template to fold into a looped configuration. The thermodynamics of the trigger binding and DNA template association are in a regime that creates a biphasic DNA amplification reaction that amplifies the reporter oligonucleotide sequence (tYp) at a non-linear amplification rate, which can demonstrate cooperative Hill kinetics. Such biphasic ultrasensitive kinetics and the templates necessary for creating such kinetics have not previously been reported.

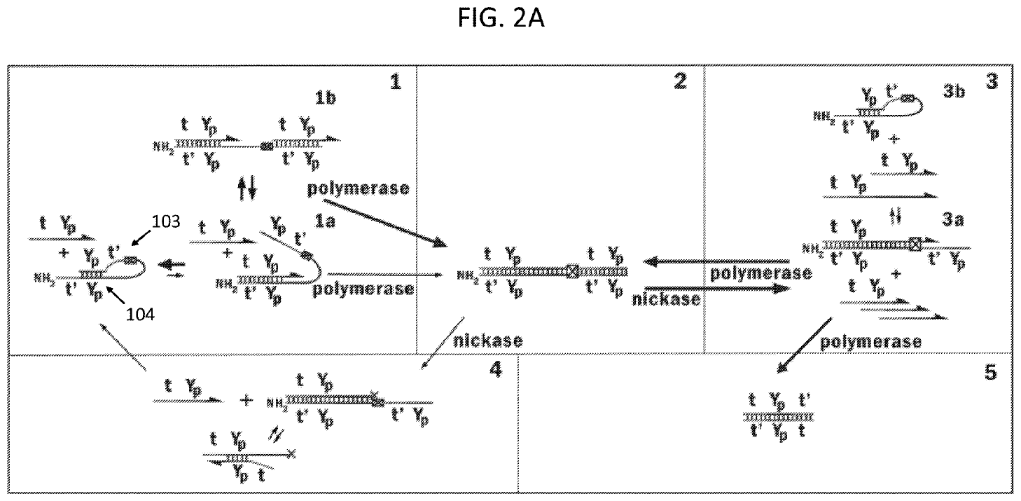

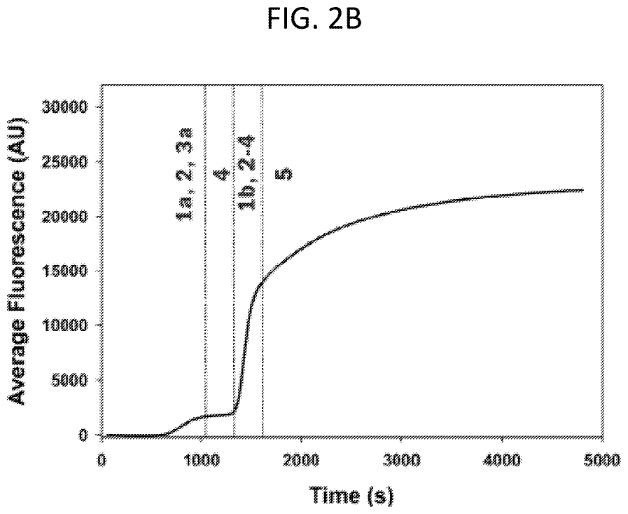

The mechanism behind the switch-like oligonucleotide amplification reaction is likely driven by multiple phenomena, as detailed in FIG. 2A. The amplification requires a looped DNA template (t′YpR2t′Yp) (having two palindromic sequences (Yp), two toeholds (t′), and a nicking enzyme recognition site ( [symbol] )), as well as polymerase and nickase enzymes. The DNA template may comprise a blocking 3′ group, such as an amine group (NH₂), to prevent extension of the template, a 3′ toehold (t′), a palindromic sequence (Yp), the nicking enzyme binding site ( [symbol] ), the repeated 5′ toehold, and the repeated palindromic sequence (panel 1). The palindromic sequences bind, forming a palindrome (104), and thus cause the template to fold into a stem and loop configuration (103). The reaction amplifies a oligonucleotide trigger sequence (tYp) with a reverse compliment to the template toehold (t′) and the palindromic region (Yp); arrows show extendable 3′ ends of the DNA. The oligonucleotide trigger (tYp) can bind to either toehold region (t′) and strand displace the palindrome region (104), thus opening the loop (shown in subpanel 1). A polymerase can then extend oligonucleotide trigger (tYp) along the DNA template and create the binding site for a nicking enzyme (shown in subpanel 2), as well as an identical trigger. The nicking enzyme (e.g., nickase) then nicks the sense strand (shown in subpanel 3a). The polymerase extends at this nick and the downstream oligonucleotide trigger (tYp) is actively or passively displaced (subpanel 3a→subpanel 2). The displaced oligonucleotide trigger (tYp) is then free to prime other templates, leading to amplification (subpanel 3a→subpanel 1). The amplification therefore produces both triggers and long triggers that contain the nickase recognition site on their 3′ end (subpanel 3b).

Still referring to FIG. 2A, the presence of the palindromic sequence produces several new reaction pathways. The palindromic region can cause trigger dimerization, after which the toehold regions can be filled by the polymerase; this removes trigger molecules from further amplification cycles (subpanel 5). The trigger can catalyze removal of the long, stable trigger by binding to either the palindromic region of the long trigger or by binding to the template, facilitating loop closure (shown in subpanels 3b, 4). Without being bound by theory, this may be vital to remove “poisoned” long triggers that cannot amplify and block further trigger amplification on the template (subpanel 4). Loop closure will also aid in removal of trigger and long trigger from the template. Finally, the presence of the loop with two toehold regions creates co-operative binding between the triggers and the looped template. For most templates, the looped configuration is more stable than the open, trigger-bound configuration (Table SI 2). The association of the template and the first trigger molecule will open the loop, which both aids and stabilizes a second trigger association (subpanel 1b). Without being bound by theory, these new reaction pathways create the unique features of our amplification reaction as detailed in FIG. 2B.

As such, in various embodiments, a method of detecting a target oligonucleotide sequence (X) is provided. In some embodiments, the method includes forming a reaction mixture that comprises: (1) a target nucleic acid comprising a target oligonucleotide sequence (X); (2) a first antisense template (X′R1t′Yp); and (3) a second antisense template (t′YpR2t′Yp). The first antisense template (X′R1t′Yp) comprises from 3′ to 5′: (a) a first sequence of nucleotides (X′) that is at least substantially complementary to the target oligonucleotide sequence (X); (b) a second sequence of nucleotides (R1) of an anti-sense strand of a first nicking enzyme binding site; and (c) a third sequence of nucleotides (t′Yp) that is at least substantially complementary to a reporter oligonucleotide sequence (tYp). The third sequence of nucleotides (t′Yp) comprises from 3′ to 5′: (i) a toehold nucleotide sequence (t′); and (ii) a palindromic nucleotide sequence (Yp). In some embodiments, the third sequence of nucleotides (t′Yp) is non-complementary to the target oligonucleotide sequence (X). The second antisense template (t′YpR2t′Yp) comprises from 3′ to 5′: (a) a fourth sequence of nucleotides comprising t′Yp; (b) a fifth sequence of nucleotides (R2) of an anti-sense strand of a second nicking enzyme binding site; and (c) a sixth sequence of nucleotides comprising t′Yp. The two palindromic nucleotide sequences (Yp) of the second antisense template (t′YpR2t′Yp) cause the second antisense template (t′YpR2t′Yp) to form a palindrome and fold into a stem and loop configuration. In some aspects of the methods, the DNA template can include more than two copies of t′Yp. For example the DNA template may contain three copies of t′Yp (forming antisense template t′YpR2t′YpR2t′Yp), four copies of t′Yp (forming antisense template t′YpR2t′YpR2t′YpR2t′Yp), five copies of t′Yp (forming antisense template t′YpR2t′YpR2t′YpR2t′Yp), etc. The reaction mixture further includes a polymerase, a first nicking enzyme that nicks the first nicking site, a second nicking enzyme that nicks the second nicking site, and nucleotides. The method further includes subjecting the reaction mixture to essentially isothermal conditions at a reaction temperature to amplify the reporter oligonucleotide sequence (tYp) at a non-linear amplification rate and detecting the reporter oligonucleotide sequence (tYp). In some aspects, the non-linear amplification rate of the reporter oligonucleotide sequence (tYp) demonstrates cooperative Hill kinetics.

In some aspects of a method of detecting a target oligonucleotide sequence (X), the amplification of the reporter oligonucleotide is biphasic. In some aspects, the first phase linearly amplifies the oligonucleotide sequence (tYp) and the second phase amplifies the reporter oligonucleotide sequence (tYp) at a non-linear amplification rate. In aspects, the non-linear amplification rate of the reporter oligonucleotide sequence (tYp) demonstrates cooperative Hill kinetics.

In some aspects of a method of detecting a target oligonucleotide sequence (X), the reporter oligonucleotide sequence (tYp) is linearly amplified from the steps including: (A) forming a duplex (D1) comprising the target oligonucleotide sequence (X) and the first antisense template (X′R1t′Yp); (B) extending, using the polymerase, the target oligonucleotide sequence (X) of the duplex (D1) along the first antisense template (X′R1t′Yp) to form an extended target oligonucleotide sequence comprising a sense sequence complementary to the first antisense template (X′R1t′Yp); (C) nicking, with the first nicking enzyme, at the first nicking enzyme binding site on the sense strand of the duplex (D1) to produce the reporter oligonucleotide sequence (tYp); and (D) repeating steps (B) and (C) to thereby linearly amplify the reporter oligonucleotide sequence (tYp).

In some aspects of a method of detecting a target oligonucleotide sequence (X), the reporter oligonucleotide (tYp) is non-linearly amplified at a non-linear amplification rate from the steps including: (A) forming a duplex (D2) comprising the reporter oligonucleotide sequence (tYp) and the second antisense template (t′YpR2t′Yp), wherein binding of the reporter oligonucleotide sequence (tYp) to the toehold site (t′) unfolds the stem and loop configuration of the second antisense template (t′YpR2t′Yp); (B) extending, using the polymerase, the reporter oligonucleotide sequence (tYp) of the duplex (D2) along the second antisense template (t′YpR2t′Yp) to form an extended reporter oligonucleotide sequence comprising a sense sequence complementary to the second antisense template (t′YpR2t′Yp); (C) nicking, with the second nicking enzyme, at the second nicking enzyme binding site on the sense strand of the duplex (D2) to produce the reporter oligonucleotide sequence (tYp); and (D) repeating steps (B) and (C) to thereby amplify the reporter oligonucleotide sequence (tYp) at a non-linear amplification rate. In aspects, the non-linear amplification rate of the reporter oligonucleotide sequence (tYp) demonstrates cooperative Hill kinetics.

In some aspects of a method of detecting a target oligonucleotide sequence (X), the reporter oligonucleotide sequence (tYp) is from 8-30 nucleotides in length. In some aspects of the previous embodiments of a method of amplifying a reporter oligonucleotide sequence (tYp), the toehold site (t′) of the first, third, fourth, and fifth sequence of nucleotides is from 3-8 nucleotides in length. In some aspects of the previous embodiments of a method of amplifying a reporter oligonucleotide sequence (tYp), the palindrome of the second antisense template (t′YpR2t′Yp) is from 4-22 nucleotides in length. In some aspects, the thermodynamic property of the antisense template (t′YpR2t′Yp) should be ≥0.5 kcal/mole, as determined by Equation 1, wherein Δ G5′ toehold is the free energy of the toehold (t′) binding on the 5′ end of the template, Δ G3′ toehold is the free energy of toehold (t′) binding on the 3′ end of the template, ΔGpalindrome is the free energy of palindrome (Yp) association, ΔGloop is the free energy of the template looped secondary structure, and ΔGtrigger:template is the free energy of trigger association with an open template (tYp:t′YpR2t′Yp complex) (as given by Mfold web server, an open source software that uses empirical free energies of DNA hybridization that have been corrected for salt concentration (http://unafold.rna.albany.edu/?q=mfold):

ΔG5′ toehold+ΔG3′ toehold+ΔGpalindrome−ΔGloop−ΔGtrigger:template (Eq. 1)

In describing the positioning of certain sequences on nucleic acid molecules, such as, for example, in the target sequence, reporter sequence, or templates, it is understood by those of ordinary skill in the art that the terms “3′” and “5′” refer to a location of a particular sequence or region in relation to another. Thus, when a sequence or a region is 3′ to or 3′ of another sequence or region, the location is between that sequence or region and the 3′ hydroxyl of that strand of nucleic acid. When a location in a nucleic acid is 5′ to or 5′ of another sequence or region, that means that the location is between that sequence or region and the 5′ phosphate of that strand of nucleic acid.

Amplification of a nucleic acid molecule or the like, as used herein, refers to use of a technique that increases the number of copies of a nucleic acid molecule (e.g., a DNA or RNA molecule, such as cDNA) in a sample. Embodiments disclosed herein include isothermal amplification of a nucleic acid molecule.

Isothermal amplification of nucleic acid molecules or the like, as used herein, refers to nucleic acid amplification methods that do not require temperature cycling for the denaturation, annealing or extension steps (though a single initial denaturation step may be included in isothermal amplification assays, for example, prior to addition of the polymerase to the assay, particularly for double stranded targets). Thus, in contrast to PCR methods, these steps are performed at a single temperature in isothermal amplification assays, whereas multiple temperatures are used in PCR assays. The nicking and the extension reaction will work at the same temperature or within the same narrow temperature range. However, it is not necessary that the temperature be maintained at precisely one temperature. If the equipment used to maintain an elevated temperature allows the temperature of the reaction mixture to vary by a few degrees (such as varying by less than 1 degree, less than 2 degrees, or less than 3 degrees), this is not detrimental to the amplification reaction, and may still be considered to be an isothermal reaction. Conditions sufficient for isothermal amplification of nucleic acid molecules using a strand displacement polymerase are familiar to the person of ordinary skill in the art.

Target nucleic acid molecule, as used herein, refers to a nucleic acid molecule whose amplification, detection, quantitation, qualitative detection, or a combination thereof, is intended. For example, the target can be a defined region or particular portion of a nucleic acid molecule, for example a portion of a genome (such as a gene or a region of DNA or RNA containing a gene (or portion thereof) of interest). A target nucleic acid could be any kind of natural or synthetic DNA molecule, including oligonucleotides, genomic DNA, mitochondrial DNA, cDNA derived from reverse transcription of mRNA, microRNA, or siRNA, and so on. The said target nucleic acid could also be any type of synthetic or natural RNA molecules, including mRNA, microRNA and siRNA, and so on. Target nucleic acid molecules include single and/or double stranded nucleic acid molecules. The target nucleic acid can be a portion of a longer nucleic acid molecule from a target organism (such as a target pathogen) or target cell (such as a cancer cell), such as a pathogenic genomic, DNA, cDNA, RNA, or mRNA sequence or a tumor-associated genomic, DNA, cDNA, RNA, or mRNA sequence. The nucleic acid molecule need not be in a purified form. Various other nucleic acid molecules can also be present with the target nucleic acid molecule. For example, the target nucleic acid molecule can be a specific nucleic acid molecule or sequence (which may be referred to herein as a target oligonucleotide sequence or target oligonucleotide and which can include RNA or DNA), the amplification of at least a portion thereof (such as a portion of a genomic sequence or cDNA sequence) is intended. Target nucleic acid molecules, including target oligonucleotide sequences contained therein, provide a 3′ hydroxyl group for an initial oligonucleotide extension. Purification or isolation of the target nucleic acid molecule, if needed, can be conducted by methods known to those in the art, such as by using a commercially available purification kit or the like. The use of the term “target sequence” or “target oligonucleotide sequence” may refer to either the sense or antisense strand of the sequence, and also refers to the sequences as they exist on target nucleic acids, amplified copies, or amplification products, of the original target sequence. The amplification product may be a larger molecule that comprises the target sequence, as well as at least one other sequence, or other nucleotides. In embodiments, the target sequence should not contain nicking sites for any nicking enzymes that will be included in the reaction mix.

In some aspects of the previous embodiments of a method of amplifying a reporter oligonucleotide sequence (tYp), the target nucleic acid comprising a target oligonucleotide sequence (X) is any synthetic or natural RNA molecule, including mRNA, microRNA, and siRNA. In other aspects of the previous embodiments, the target nucleic acid comprising a target oligonucleotide sequence (X) is any synthetic or natural DNA molecule, including genomic DNA, mitochondrial DNA, cDNA derived from reverse transcription of mRNA, microRNA, or siRNA, and said method comprises a step of denaturing and/or nicking/cleaving said target nucleic acid comprising a target oligonucleotide sequence (X) prior to forming the reaction mixture so that a single stranded target nucleic acid comprising a target oligonucleotide sequence (X) is formed.

Nucleic acid, as used herein, refers to a polymer composed of nucleotide units (ribonucleotides, deoxyribonucleotides, related naturally occurring structural variants, and synthetic non-naturally occurring analogs thereof) linked via phosphodiester bonds, related naturally occurring structural variants, and synthetic non-naturally occurring analogs thereof. Thus, the term includes nucleotide polymers in which the nucleotides and the linkages between them include non-naturally occurring synthetic analogs, such as, for example and without limitation, phosphorothioates, phosphoramidates, methyl phosphonates, chiral-methyl phosphonates, 2-O-methyl ribonucleotides, peptide-nucleic acids (PNAs), and the like. Such polynucleotides can be synthesized, for example, using an automated DNA synthesizer. It will be understood that when a nucleotide sequence is represented by a DNA sequence (i.e., A, T, G, C), this also includes an RNA sequence (i.e., A, U, G, C) in which “U” replaces “T.”

Nucleotide, as used herein, includes (but is not limited to), a monomer that includes a base linked to a sugar, such as a pyrimidine, purine or synthetic analogs thereof, or a base linked to an amino acid, as in a peptide nucleic acid (PNA). A nucleotide is one monomer in a polynucleotide. A “native nucleotide” refers to adenylic acid, guanylic acid, cytidylic acid, thymidylic acid, or uridylic acid. A “derivatized nucleotide” is a nucleotide other than a native nucleotide. The reaction methods may be conducted in the presence of native nucleotides. The reaction may also be carried out in the presence of labeled nucleotides (such as native nucleotides), such as, nucleotides linked to or including, for example, radiolabels such as, for example, ³2P, ³³P, ¹²⁵I or ³⁵S, enzyme labels such as alkaline phosphatase, fluorescent labels such as fluorescein isothiocyanate (FITC), biotin, avidin, digoxigenin, antigens, haptens, or fluorochromes. These derivatized nucleotides may, for example, be present in the templates or reporter oligonucleotide. A nucleotide sequence refers to the sequence of bases in a polynucleotide.

Oligonucleotide, as used herein, is a linear polynucleotide sequence comprising two or more deoxyribonucleotides or ribonucleotides, e.g., more than three.

The target sequences, e.g., target nucleic acid comprising a target oligonucleotide sequence (X), may be amplified in many types of samples including, but not limited to samples containing spores, viruses, cells, nucleic acid from prokaryotes or eukaryotes, or any free nucleic acid. The sample may be isolated from any material suspected of containing the target sequence. For example, the sample may be a biological sample. Biological sample, as used herein, refers to a sample including biological material, for example a sample obtained from a subject, or an environmental sample containing biological material. For example, biological samples include all clinical samples useful for detection of disease or infection in subjects. For animals, for example, mammals, such as, for example, humans, the sample may comprise blood, serum, bone marrow, mucus, lymph, hard tissues, for example, liver, spleen, kidney, lung, or ovary, biopsies, sputum, saliva, tears, feces, or urine. In aspects, the target sequence may be present in air, plant, soil, or other materials suspected of containing biological organisms. As such, in some aspects of the previous embodiments of a method of detecting a target oligonucleotide sequence (X), the target nucleic acid comprising a target oligonucleotide sequence (X) is obtained from a sample derived from an animal. In some aspects, the sample is blood, serum, mucus, saliva, urine, or feces. In other aspects, a target oligonucleotide sequence (X) is obtained from a sample derived form a microbe, a virus, a fungus, an insect, or a plant.

One of ordinary skill in the art will know suitable methods for extracting nucleic acids such as RNA and/or DNA from a sample. Such methods will depend upon, for example, the type of sample in which the nucleic acid is found. Nucleic acids can be extracted using standard methods. For example, rapid nucleic acid preparation can be performed using a commercially available kit (such as kits and/or instruments from Qiagen (such as DNEasy® or RNEasy® kits), Roche Applied Science (such as MagNA Pure kits and instruments), Thermo Scientific (KingFisher mL), bioMérieux (Nuclisens® NASBA Diagnostics), or Epicentre (Masterpure™ kits)). In other examples, the nucleic acids may be extracted using guanidinium isothiocyanate, such as single-step isolation by acid guanidinium isothiocyanate-phenol-chloroform extraction (Chomczynski et al. Anal. Biochem. 162:156-159, 1987). The sample can be used directly or can be processed, such as by adding solvents, preservatives, buffers, or other compounds or substances. In still other examples, no extraction procedure is performed prior to the amplification reaction. In still other examples, cell lysis is performed prior to the amplification reaction.

The term “complementary” as it refers to two nucleic acid sequences generally refers to the ability of the two sequences to form sufficient hydrogen bonding between the two nucleic acids to stabilize a double-stranded nucleotide sequence formed by hybridization of the two nucleic acids. A first nucleic acid is “at least substantially complementary” to a second nucleic acid sequence when the first sequence is able to hybridize or bind to the second sequence to form at last a transient duplex under the reaction conditions (e.g., essentially isothermal conditions at a reaction temperature). For example, in some embodiments, a first nucleic acid is “at least substantially complementary” to a second nucleic acid sequence acid molecule, such as at least 90% of the first nucleic acid is complementary to the corresponding region of the second nucleic acid sequence (for example at least 91%, 92%, 93%, 94%, 95%, 96%, 97%, 98% or 99% complementary to the corresponding region). In some aspects of the embodiments of a method of amplifying a reporter oligonucleotide sequence (tYp), the first sequence of nucleotides (X′) is completely complementary to the target oligonucleotide sequence (X). In some aspects of method of detecting a target oligonucleotide sequence (X), the third sequence of nucleotides (t′Yp) is completely complementary to the reporter oligonucleotide sequence (tYp). Completely complementary means that a first sequence is exactly complementary to a second sequence, that is, each nucleotide of the first sequence is complementary to the nucleotide of the second sequence at its corresponding position, and the first sequence is the same length as the second sequence.

“Nicking” refers to the cleavage of only one strand of the double-stranded portion of a fully or partially double-stranded nucleic acid. The position where the nucleic acid is nicked is referred to as the nicking site or nicking enzyme site. The recognition sequence that the nicking enzyme recognizes is referred to as the nicking enzyme binding site. “Capable of nicking” refers to an enzymatic capability of a nicking enzyme.

A nicking enzyme is a protein that binds to double-stranded nucleic acids (e.g., DNA, RNA, DNA/RNA hybrid etc.) and cleaves one strand of a double-stranded duplex. A nicking enzyme may cleave either upstream or downstream of the binding site, or nicking enzyme recognition site, as a result of which a so-called nick is inserted into the double-stranded nucleic acid, in which the 5′-3′ phosphodiester bond between two nucleotides is hydrolytically cleaved. The nicking enzyme thus acts as a phosphodiesterase, so that a single-strand break is inserted in the double strand and a free 3′-OH end is created, which serves as an attachment point for a polymerase. In embodiments of the disclosed processes, only one strand of the double strand (e.g., Duplex 1 or Duplex 2) is cleaved, namely the forward, top, or sense strand, on which the binding site sequence for the nicking enzyme is also situated, and the other strand remains intact. Nicking enzymes which cleave not only one strand of the double strand, but instead both, are generally not suitable for use in the reaction mixture of the method according to the invention. In exemplary embodiments, the reaction comprises the use of nicking enzymes that cleave or nick downstream of the binding site (top strand nicking enzymes) so that the product sequence does not contain the nicking site. Using an enzyme that cleaves downstream of the binding site may allow the polymerase to more easily extend without having to displace the nicking enzyme.

Examples of suitable nicking enzymes, include, but are not limited to, Nt.BstNBI, Nt.BspQI, Nb.BbvCI, Nb.BsmI, Nb.BsrDI, Nb.BtsI, Nt.AlwI, Nt.BbvCI, Nt.CviPII, Nt.BsmAI, Nb.Bpu10I and Nt.Bpu10I. Further suitable nicking enzymes are familiar to those skilled in the art. In some aspects of a method of detecting a target oligonucleotide sequence (X), the nicking enzyme is selected from the group consisting of Nt.BstNBI, Nt.BspQI, Nb.BBvCl, Nb.Bsml, Nb.BsrDI, Nb.BstI, Nt.AlwI, Nt.BbvCI, Nt.CviPII, Nt.BsmAI, Nb.Bpu1oI, and Nt.Bpu10I.

The nicking binding site sequence of the templates depends on which nicking enzyme is chosen for each template (e.g., the transduction template and DNA template). Different nicking enzymes may be used in a single assay, but a simple amplification may, for example, employ a single nicking enzyme for use with both templates. Thus, the embodiments of the present invention include those where both templates comprise enzyme binding sites for the same nicking enzyme, and only one nicking enzyme is used in the reaction. In these embodiments, both the first and second nicking enzymes are the same. The present invention also includes those embodiments where each template comprises a nicking enzyme binding site for a different nicking enzyme, and two different nicking enzymes are used in the reaction. In some aspects of a method of detecting a target oligonucleotide sequence (X), the first nicking binding site is identical to the second nicking binding site. In some aspects of a method of detecting a target oligonucleotide sequence (X), the first nicking binding site and the second nicking binding site are nicked by the same nicking enzyme.

The nick in the double-stranded amplification products is recognized by a polymerase. A polymerase in the sense of the present invention is an enzyme for nucleic acid replication and/or for nucleic acid repair. The polymerase fills the nick at the 3′-OH end beginning with nucleotides which are complementary to the template strand. For this, e.g., a deoxyribonucleotide phosphate corresponding to the complementary base is successively attached each time and incorporated via a phosphodiester bond with elimination of pyrophosphates. In embodiments, the polymerization reaction takes place in the 5′→3′ direction. In embodiments, the polymerase may possess no 5′→3′ exonuclease activity and/or also have a strand displacement activity. In embodiments, the polymerase may be thermophilic so that it is active at an elevated reaction temperature. In certain embodiments, the polymerase is a warm start polymerase. If the polymerase also has the capability of extending an RNA primer (such as Bst (large fragment), 9° N, Therminator, Therminator II, etc.) the reaction can also amplify RNA targets in a single step without the use of a separate reverse transcriptase.

In some embodiments of the disclosed methods, the polymerase is one with strand displacement activity. In some embodiments of the disclosed methods, the polymerase does not need to have stand displacement activity, and the the reporter oligonucleotide sequence (tYp) can be released passively from the templates (e.g., transduction template and DNA template) after nicking. Examples of suitable polymerases include, but are not limited to, Bst DNA polymerase, Bst 2.0 WarmStart® DNA Polymerase, Bst DNA polymerase (Large fragment), 9° Nm DNA polymerase, Phi29 DNA polymerase, DNA polymerase I (E. coli), DNA polymerase I, Large (Klenow) fragment, Klenow fragment (3′-5′ exo-), T4 DNA polymerase, T7 DNA polymerase, Deep VentR™ (exo-) DNA Polymerase, Deep VentR™ DNA Polymerase, DyNAzyme™ EXT DNA, DyNAzyme™ II Hot Start DNA Polymerase, Phusion™ High-Fidelity DNA Polymerase, Therminator™ DNA Polymerase, Therminator™ II DNA Polymerase, VentR®DNA Polymerase, VentR® (exo-) DNA Polymerase, RepliPHFM Phi29 DNA Polymerase, rBst DNA Polymerase, Large Fragment (IsoTherm™ DNA Polymerase), MasterAmp™ AmpliTherm™ DNA Polymerase, Tag DNA polymerase, Tth DNA polymerase, Tfl DNA polymerase, Tgo DNA polymerase, SP6 DNA polymerase, Tbr DNA polymerase, DNA polymerase Beta, ThermoPhi DNA polymerase. Further suitable polymerases are known to those skilled in the art.

The templates of the present invention may include, for example, spacers, blocking groups, and modified nucleotides. Modified nucleotides are nucleotides or nucleotide triphosphates that differ in composition and/or structure from natural nucleotide and nucleotide triphosphates. Modified nucleotide or nucleotide triphosphates used herein may, for example, be modified in such a way that, when the modifications are present on one strand of a double-stranded nucleic acid where there is a restriction endonuclease recognition site, the modified nucleotide or nucleotide triphosphates protect the modified strand against cleavage by restriction enzymes. Thus, the presence of the modified nucleotides or nucleotide triphosphates encourages the nicking rather than the cleavage of the double-stranded nucleic acid. Blocking groups are chemical moieties that can be added to the template to inhibit target sequence-independent nucleic acid polymerization by the polymerase. Blocking groups are usually located at the 3′ end of the template. Non-limiting examples of blocking groups include, for example, amine groups (NH₂), alkyl groups, non-nucleotide linkers, phosphorothioate, alkane-diol residues, peptide nucleic acid, and nucleotide derivatives lacking a 3′-OH, including, for example, cordycepin. Examples of spacers, include, for example, C3 spacers. Spacers may be used, for example, within the template, and also, for example, at the 5′ end, to attach other groups, such as, for example, labels. In some aspects of the previous embodiments of a method of amplifying a reporter oligonucleotide sequence (tYp), the 3′ terminus of the first antisense template (X′R1t′Yp) and the 3′ terminus of the second antisense template (t′YpR2t′Yp) are blocked.

Conditions sufficient for isothermal amplification of nucleic acid molecules, for example, using a strand displacement polymerase are familiar to the person of ordinary skill in the art. For example, buffer conditions are disclosed in Notami et al., Nucl. Acid. Res., 28:e63, 2000 and U.S. Pat. No. 8,017,357. Suitable buffers can also be obtained from the manufacturer or supplier of a particular polymerase. Is some embodiments, an aqueous buffer is used (e.g., but not limited to Tris buffer, a phosphate buffer, or a carbonate buffer, as is known in the art). An aqueous buffer may have a pH of about 7-about 9. The buffer may contain monovalent (e.g. Na⁺, typically used at 30-200 mM) and/or divalent (Ca⁺⁺ or Mg⁺⁺ salts, typically used at 2-20 mM). The polymerase may be mixed with the target nucleic acid molecule before, after, or at the same time as, the nicking enzyme. In exemplary embodiments, a reaction buffer is optimized to be suitable for both the nicking enzyme and the polymerase, as is familiar to the person of ordinary skill in the art. Further, variations in buffer conditions, salt concentration (e.g., MgSO₄, KCl), polymerase concentration, nicking enzyme concentration, template concentrations, and free nucleotide concentration all can be optimized based on the assay sequence and desired detection method, which is within the skill of the person of ordinary skill in the art.

The amplification reaction is typically carried out at about 37-75° C., such as about 37-70, 37-42, 54-70, 55-65, 55-60, 60-77, 60-70, 60-65 or 65-70° C. In some embodiments, the amplification reaction is carried out at about 54-75° C., such as about 54, 55, 56, 57, 58, 59, 60, 61, 62, 63, 64, 65, 66, 67, 68, 69, 70, 71, 72, 73, 74 or 75° C. The reaction is run at a substantially constant temperature, usually between 54° C. and 60° C., such as 55° C. for the enzyme combination of Nt.BstNBI nicking endonuclease, Bst 2.0 WarmStart® DNA Polymerase. Other enzyme combinations may be used and the optimal reaction temperature will be based on the optimal temperature for both the nicking enzyme and polymerase to work in concert as well as the melting temperature of the reaction products. In some aspects of the previous embodiments of a method of amplifying a reporter oligonucleotide sequence (tYp), the amplification of the reporter oligonucleotide sequence (tYp) is performed at about 55° C. to about 60° C.

In some aspects of a method of detecting a target oligonucleotide sequence (X), the palindrome of the second antisense template (t′YpR2t′YP) has a melting temperature that is greater than the reaction temperature, but less than 90° C. In other aspects, the second antisense template (t′YpR2t′YP) has a melting temperature that is greater than the reaction temperature, but less than 89° C., 88° C., 87° C., 86° C., 85° C., 84° C., 83° C., 82° C., 81° C., or 80° C. In some aspects of a method of detecting a target oligonucleotide sequence (X), the duplex (D2) has a melting temperature that is less than the reaction temperature plus 5° C.

In some embodiments, particularly when target nucleic acid comprising a target oligonucleotide sequence (X) is double stranded, the amplification method includes an initial denaturation step, for example but not limited to, wherein the reaction mixture is heated to about 95° C. for about five minutes before addition of the strand displacement polymerase to the reaction mixture. Similarly, if the target nucleic acid comprising a target oligonucleotide sequence (X) is double stranded, the double stranded target may be cleaved on both strands (e.g., using an appropriate restriction enzyme) to separate the target to become single stranded prior to running the instantly-disclosed reaction.

The template concentrations are typically in excess of the concentration of target. The concentrations of the transduction and DNA templates can be at the same or at different concentrations to bias the amplification of one product over the other. The concentration of each is usually between 10 nM and 1 μM (for example, about 10 nM, about 25 nM, about 50 nM, about 100 nM, about 250 nM, about 500 nM, about 1 μM, such as about 10-100 nM, about 25-250 nM, about 50-500 nM, about 100-500 nM, about 250-750 nM, or about 500 nm to 1 μM).

Additives, such as, but not limited to, BSA, non-ionic detergents such as Triton X-100 or Tween-20, DMSO, DTT, and RNase inhibitor may be included for optimization purposes without adversely affecting the amplification reaction.

The period of time that the isothermal amplification reaction is allowed to proceed can vary according to the particular conditions of the reaction. For example, in some embodiments, amplifying a target oligonucleotide sequence (X) includes contacting the a target oligonucleotide sequence (X) with the instantly-disclosed reaction mixture under conditions sufficient for isothermal amplification for a period of at least 5 minutes such as about 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 75, 90 about 120 minutes, about 150 minutes, about 180 minutes or any value or range therebetween (for example, 5-60 minutes, 30-90 minutes, 60-120 minutes, 80-160 minutes, or 90-180 minutes). In additional examples, the period of time can be from about 10-60 minutes, such as about 10-15, 10-20, 10-30, 15-20, 15-30, 20-30, 30-60 minutes, or any value or range therebetween. In several examples, the isothermal amplification reaction is allowed to proceed for a maximum period of time, such as no more than 90 minutes, for example about 10, 15, 20, 25, 30, 45, 60, 75 or 90 minutes.

Reactions may be allowed to proceed to completion, that is, when one of the resources is exhausted. Or, the reaction may be stopped using methods known to those of ordinary skill in the art, such as, for example, heat denaturation, or the addition of EDTA, high salts, or detergents. In exemplary embodiments, where mass spectrometry is to be used following amplification, EDTA may be used to stop the reaction.

In some embodiments, more than one target oligonucleotide sequence (X) is detected in a multiplexing approach. The sequence of the templates (transduction template and DNA template, including the reporter oligonucleotide sequence) differ in their nucleotide sequence, so that each set of templates is specific for their target oligonucleotide sequence (X) to the extent that in the multiplexing approach several different target oligonucleotide sequences can be detected simultaneously in one reaction vessel. In some examples, the methods include detecting two or more target oligonucleotide sequences (such as 2, 3, 4, 5, 6, 7, 8, 9, 10, or more target oligonucleotide sequences) in a single reaction vessel.

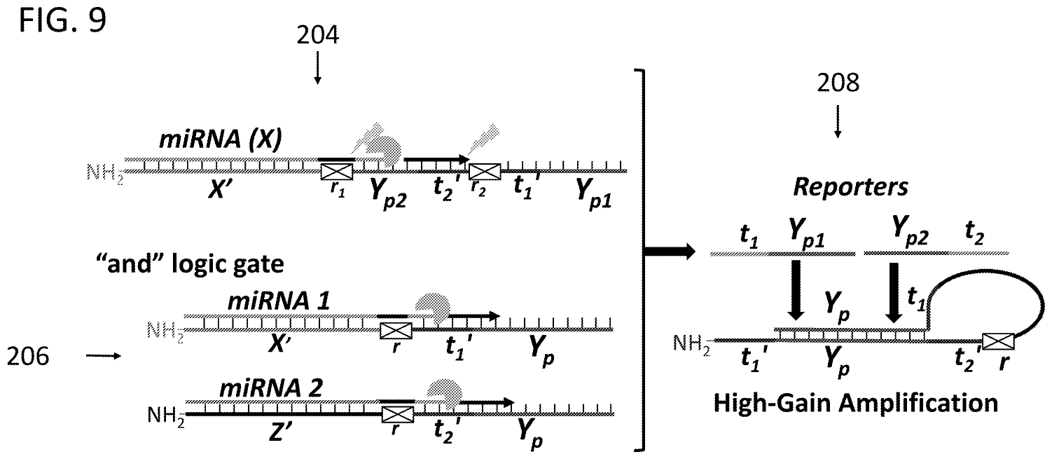

In some aspects, such a multiplexing approach can be used to create an “OR” logic gate. This can occur by using multiple DNA templates, each specific for a different target oligonucleotide sequence (X), that each transduce the different oligonucleotide sequences into the same reporter oligonucleotide sequence (tYp). The generated reporter oligonucleotide sequence (tYp) would initiate reporter oligonucleotide sequence (tYp) amplification as shown in FIGS. 1A-B and FIG. 2A. In other aspects, such a multiplexing approach can be used to create an “AND” logic gate. This is shown in FIG. 9. Splitting the reporter molecule could enhance the cooperative hill kinetic effect on DNA template loop opening, and ensure that the logic gate will not turn on without input form two different sources. FIG. 9 depicts a splitting the reporter molecule using a single transduction template (204) so that two reporters, t₁Yp₁ and t₂Yp₂, are produced (208), as well as generating two reporter molecules using two transduction templates (206) so that two reporters, t₁Yp₁ and t₂Yp₂, are produced (208).

The methods disclosed herein can be carried out in various formats. For example, the reactions may be performed in a mixture where all the components are soluble. Alternatively, one or all of the template(s) may be bound to a solid phase; for example, the 3′ or 5′ end may be covalently attached to a solid phase with the use of cross-linkers or spacers. Solid phases may include, by way of example, beads, microbeads, microplates, microplate wells, membranes, slides, and arrays. Materials of such solid phases may include, by way of example, glass, nylon, silica, and plastics. The templates may also be fixed in a gene chip or array, so that a large number of different target oligonucleotides can be detected by a high throughput method. Such a matrix can be made by various materials including silicon materials such as silicon or silicon dioxide film, silicon substrate, silicon nanowires, conductive metals such as gold, platinum, carbon materials, such as graphite, carbon nanotubes, and conductive resin, and so on. The methods disclosed herein can also be carried out in a microfluidic format.

In some aspects of a method of detecting a target oligonucleotide sequence (X), the method further includes the step of detecting the amplified reporter oligonucleotide sequence (tYp). In some aspects, the amplified reporter oligonucleotide sequence may be detected by any method known to one of skill in the art. Non-limiting examples include, but are not limited to luminescence spectroscopy or spectrometry, fluorescence, fluorescence spectroscopy or spectrometry, mass spectrometry, liquid chromatography, fluorescence polarization, colorimetry, and electrophoresis.

In some aspects of the methods of detecting a target oligonucleotide sequence (X), the step of detecting the amplification rate of the reporter oligonucleotide sequence (tYp) is performed at least partially by a technique selected from the group consisting of luminescence spectroscopy or spectrometry, fluorescence, fluorescence spectroscopy or spectrometry, mass spectrometry, liquid chromatography, fluorescence polarization, colorimetry, and electrophoresis

In one example, the amplified reporter oligonucleotide sequence (tYp) may be detected by gel electrophoresis, thus detecting amplified reporter oligonucleotide sequence (tYp) that has a specific size. Further, one or more of the nucleotides included in the reaction may be, for example, labeled with biotin. Biotin-labeled amplified sequences may be captured using avidin bound to a signal generating enzyme, for example, peroxidase.

In another example, the disclosed amplification reaction may be carried out in the presence of a labeled nucleoside (e.g., labeled deoxnucleoside triphosphate) so that the label in incorporated into the amplified reporter oligonucleotide sequence (tYp). Labels suitable for incorporating into a nucleic acid fragment and methods for the subsequent detection of the fragments are known in the art, and exemplary labels include, but are not limited to, a radiolabel for example, ³2P, ³³P, ¹²⁵I, or ³⁵S, enzyme labels such as alkaline phosphatase, fluorescent labels such as fluorescein isothiocyanate (FITC), biotin, avidin, digoxigenin, antigens, haptens, or fluorochromes.

Alternatively, amplified reporter oligonucleotide sequence (tYp) may be detected by the use of a labeled detector oligonucleotide that is substantially, and in some examples, completely complementary to the amplified reporter oligonucleotide sequence (tYp). Similar to a labeled nucleoside (e.g., a labeled deoxynucleoside triphosphate), the detector oligonucleotide may also be labeled with a hapten, antigen, enzyme, radioactive, chemiluminescent, or fluorescent tag.

Detection methods may employ the use of dyes that specifically stain a nucleic acid, such as double-stranded DNA. Intercalating dyes that exhibit enhanced fluorescence upon binding to DNA or RNA are a basic tool in molecular and cell biology. Dyes may be, for example, DNA or RNA intercalating fluorophores and may include but are not limited to the following examples: Acridine orange, ethidium bromide, Hoechst dyes, PicoGreen, propidium iodide, SYBR I (an asymmetrical cyanine dye), SYBR II, TOTO (a thiazole orange dimer) and YOYO (an oxazole yellow dimer). Dyes provide an opportunity for increasing the sensitivity of nucleic acid detection when used in conjunction with various detection methods and may have varying optimal usage parameters. For example ethidium bromide is commonly used to stain DNA in agarose gels after gel electrophoresis and during PCR (Hiquchi et al., Nature Biotechnology 10; 413-417, April 1992), propidium iodide and Hoechst 33258 are used in flow cytometry to determine DNA ploidy of cells, SYBR Green 1 has been used in the analysis of double-stranded DNA by capillary electrophoresis with laser induced fluorescence detection and Pico Green has been used to enhance the detection of double-stranded DNA after matched ion pair polynucleotide chromatography (Singer et al., Analytical Biochemistry 249, 229-238 1997).

Methods of detecting and/or continuously monitoring the amplification of nucleic acid products are also well known to those skilled in the art. Examples include the use of Molecular Beacons, Fluorescence resonance energy transfer (FRET). Molecular Beacons are hair-pin shaped oligonucleotides containing a fluorophore on one end and a quenching dye on the opposite end. The loop of the hair-pin contains a probe sequence that is complementary to a target sequence and the stem is formed by annealing of complementary arm sequences located on either side of the probe sequence. A fluorophore and a quenching molecule are covalently linked at opposite ends of each arm. Under conditions that prevent the oligonucleotides from hybridizing to its complementary target or when the molecular beacon is free in solution the fluorescent and quenching molecules are proximal to one another preventing fluorescence resonance energy transfer (FRET). When the molecular beacon encounters a target molecule, hybridization occurs; the loop structure is converted to a stable more rigid conformation causing separation of the fluorophore and quencher molecules leading to fluorescence (Tyagi et al. Nature Biotechnology 14: March 1996, 303-308). Due to the specificity of the probe, the generation of fluorescence is exclusively due to the synthesis of the intended amplified product.

Molecular beacons are extraordinarily specific and can discern a single nucleotide polymorphism. Molecular beacons can also be synthesized with different colored fluorophores and different target sequences, enabling several products in the same reaction to be quantitated simultaneously. For quantitative amplification processes, molecular beacons can specifically bind to the amplified target following each cycle of amplification, and because non-hybridized molecular beacons are dark, it is not necessary to isolate the probe-target hybrids to quantitatively determine the amount of amplified product. The resulting signal is proportional to the amount of amplified product. This can be done in real time. The specific reaction conditions may be optimized for each primer/probe set, for example, to increase accuracy and precision.

The production or presence of target nucleic acids and nucleic acid sequences may also be detected and monitored by Fluorescence resonance energy transfer (FRET). FRET is an energy transfer mechanism between two chromophores: a donor and an acceptor molecule. Briefly, a donor fluorophore molecule is excited at a specific excitation wavelength. The subsequent emission from the donor molecule as it returns to its ground state may transfer excitation energy to the acceptor molecule through a long range dipole-dipole interaction. The intensity of the emission of the acceptor molecule can be monitored and is a function of the distance between the donor and the acceptor, the overlap of the donor emission spectrum and the acceptor absorption spectrum and the orientation of the donor emission dipole moment and the acceptor absorption dipole moment. FRET is a useful tool to quantify molecular dynamics, for example, in DNA-DNA interactions as seen with Molecular Beacons. For monitoring the production of a specific product a probe can be labeled with a donor molecule on one end and an acceptor molecule on the other. Probe-target hybridization brings a change in the distance or orientation of the donor and acceptor and FRET change is observed. (Joseph R. Lakowicz, “Principles of Fluorescence Spectroscopy”, Plenum Publishing Corporation, 2nd edition (Jul. 1, 1999)).

The production or presence of target nucleic acids and nucleic acid sequences may also be detected and monitored by Mass Spectrometry. Mass Spectrometry is an analytical technique that may be used to determine the structure and quantity of the target nucleic acid species and can be used to provide rapid analysis of complex mixtures. Following the method, samples are ionized, the resulting ions separated in electric and/or magnetic fields according to their mass-to-charge ratio, and a detector measures the mass-to-charge ratio of ions. (Crain, P. F. and McCloskey, J. A., Current Opinion in Biotechnology 9: 25-34 (1998)). Mass spectrometry methods include, for example, MALDI, MALDIITOF, or Electrospray. These methods may be combined with gas chromatography (GC/MS) and liquid chromatography (LC/MS). MS has been applied to the sequence determination of DNA and RNA oligonucleotides (Limbach P., MassSpectrom. Rev. 15: 297-336 (1996); Murray K., J. Mass Spectrom. 31: 1203-1215 (1996)). MS and more particularly, matrix-assisted laser desorption/ionization MS (MALDI MS) has the potential of very high throughput due to high-speed signal acquisition and automated analysis off solid surfaces. It has been pointed out that MS, in addition to saving time, measures an intrinsic property of the molecules, and therefore yields a significantly more informative signal (Koster H. et al., Nature Biotechnol., 14: 1123-1128 (1996)).

The production or presence of target nucleic acids and nucleic acid sequences may also be detected and monitored by various methods of gel electrophoresis. Gel electrophoresis involves the separation of nucleic acids through a matrix, generally a cross-linked polymer, using an electromotive force that pulls the molecules through the matrix. Molecules move through the matrix at different rates causing a separation between products that can be visualized and interpreted via anyone of a number of methods including but not limited to; autoradiography, phosphorimaging, and staining with nucleic acid chelating dyes (e.g., DNA interchelating dyes).

The production or presence of target nucleic acids and nucleic acid sequences may also be detected and monitored by capillary gel electrophoresis. Capillary-gel Electrophoresis (CGE) is a combination of traditional gel electrophoresis and liquid chromatography that employs a medium such as polyacrylamide in a narrow bore capillary to generate fast, high-efficient separations of nucleic acid molecules with up to single base resolution. CGE is commonly combined with laser induced fluorescence (LIF) detection where as few as six molecules of stained DNA can be detected. CGE/LIF detection generally involves the use of fluorescent DNA intercalating dyes including ethidium bromide, YOYO and SYBR Green 1, but can also involve the use of fluorescent DNA derivatives where the fluorescent dye is covalently bound to the DNA. Simultaneous identification of several different target sequences can be made using this method, for example, using different fluorescent dyes for each target sequence and/or reporter oligonucleotide sequence (tYp), and/or based on size differences between the target sequences and/or reporter oligonucleotide sequence (tYp).

The production or presence of target nucleic acids and nucleic acid sequences may also be detected and monitored by various surface capture methods. This is accomplished by the immobilization of specific oligonucleotides to a surface producing a biosensor that is both highly sensitive and selective. Surfaces used in this method may include but are not limited to gold and carbon and may use a number of covalent or noncovalent coupling methods to attach the probe to the surface. The subsequent detection of a target DNA can be monitored by a variety of methods.

Electrochemical methods generally involve measuring the cathodic peak of intercalators, such as methylene blue, on the DNA probe electrode and visualized with square wave voltammograms. Binding of the target sequence can be observed by a decrease in the magnitude of the voltammetric reduction signals of methylene blue as it interacts with dsDNA and ssDNA differently reflecting the extent of the hybrid formation.

Surface Plasmon Resonance (SPR) can also be used to monitor the kinetics of probe attachment as well as the process of target capture. SPR does not require the use of fluorescence probes or other labels. SPR relies on the principle of light being reflected and refracted on an interface of two transparent media of different refractive indexes. Using monochromatic and p-polarized light and two transparent media with an interface comprising a thin layer of gold, total reflection of light is observed beyond a critical angle, however the electromagnetic field component of the light penetrates into the medium of lower refractive index creating an evanescent wave and a sharp shadow (surface plasmon resonance). This is due to the resonance energy transfer between the wave and the surface plasmons. The resonance conditions are influenced by the material absorbed on the thin metal film and nucleic acid molecules, proteins and sugars concentrations are able to be measured based on the relation between resonance units and mass concentration.

The production or presence of target nucleic acids and nucleic acid sequences may also be detected and monitored by lateral flow devices. Lateral Flow devices are well known. These devices generally include a solid phase fluid permeable flow path through which fluid flows through by capillary force. Examples include, but are not limited to, dipstick assays and thin layer chromatographic plates with various appropriate coatings. Immobilized on the flow path are various binding reagents for the sample, binding partners or conjugates involving binding partners for the sample and signal producing systems. Detection of samples can be achieved in several manners; enzymatic detection, nanoparticle detection, colorimetric detection, and fluorescence detection, for example. Enzymatic detection may involve enzyme-labeled probes that are hybridized to complementary nucleic acid targets on the surface of the lateral flow device. The resulting complex can be treated with appropriate markers to develop a readable signal. Nanoparticle detection involves bead technology that may use colloidal gold, latex and paramagnetic nanoparticles. In one example, beads may be conjugated to an anti-biotin antibody. Target sequences may be directly biotinylated, or target sequences may be hybridized to a sequence specific biotinylated probes. Gold and latex give rise to colorimetric signals visible to the naked eye and paramagnetic particles give rise to a non-visual signal when excited in a magnetic field and can be interpreted by a specialized reader.

Fluorescence-based lateral flow detection methods are also known, for example, dual fluorescein and biotin-labeled oligo probe methods, UPT-N ALP utilizing up-converting phosphor reporters composed of lanthanide elements embedded in a crystal (Corstjens et al., Clinical Chemistry, 47:10, 1885-1893, 2001), as well as the use of quantum dots.

Nucleic acids can also be captured on lateral flow devices. Means of capture may include antibody-dependent and antibody-independent methods. Antibody-dependent capture generally comprises an antibody capture line and a labeled probe of complementary sequence to the target. Antibody-independent capture generally uses non-covalent interactions between two binding partners, for example, the high affinity and irreversible linkage between a biotinylated probe and a streptavidin line. Capture probes may be immobilized directly on lateral flow membranes. Both antibody dependent and antibody independent methods may be used in multiplexing.

The production or presence of target nucleic acids and nucleic acid sequences may also be detected and monitored by multiplex DNA sequencing. Multiplex DNA sequencing is a means of identifying target DNA sequences from a pool of DNA. The technique allows for the simultaneous processing of many sequencing templates. Pooled multiple templates can be resolved into individual sequences at the completion of processing. Briefly, DNA molecules are pooled, amplified and chemically fragmented. Products are fractionated by size on sequencing gels and transferred to nylon membranes. The membranes are probed and autoradiographed using methods similar to those used in standard DNA sequencing techniques (Church et al., Science 1998 Apr. 8; 240(4849):185-188). Autoradiographs can be evaluated and the presence of target nucleic acid sequence can be quantitated.

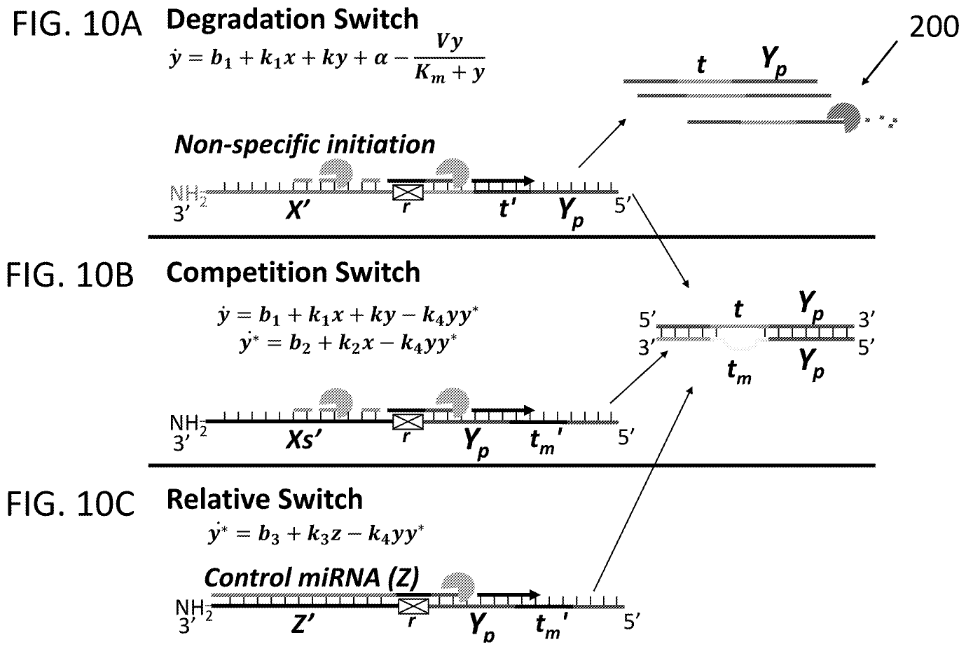

In some aspects of the instantly-disclosed methods, the methods can further include threshold-based detection, in which a signal does not turn on unless the target oligonucleotide sequence (X) is above or below a threshold concentration. Such a design is disclosed in FIGS. 10A-D. This can be used to remove non-specific signals and false positives, which are common in isothermal reactions, as previously described. It can also be used to turn on a sensor when a molecule (e.g., but not limited to, RNA mRNA, and/or miRNA) is above or below the concentration of a housekeeping gene. This is important, for example, as miRNA and mRNA are typically measured relative to a housekeeping gene, with lack of standardization being a cited concern in emerging miRNA analysis methods. Thresholding can also only turn a sensor on when a biomarker (which can be a target oligonucleotide sequence (X)) is above or below a critical concentration that indicates a malady. FIG. 10A depicts an inhibition switch design, in which the reporter strand (depicted as tYp) is degraded at a fixed rate by an exonuclease (200) that is specific for a single-stranded DNA molecule. FIG. 10B depicts a competition switch design, in which the presence of a partially complementary miRNA (Xs) in a second transduction template molecule (Xs′rYtₘ) will produce a decoy molecule (Ytₘ) that is complementary to reporter oligonucleotide sequence (tYp) (rate k₂x). The sequence Xs' is a scrambled version of X′, and can be partially complementary to endogenous RNA or varied in length to tune kinetics. This transduction template will also produce decoy molecule (Ytₘ) non-specifically (rate b₂). The decoy molecule (Yptₘ) will inactivate the reporter oligonucleotide sequence (tYp) before it undergoes amplification at a non-linear amplification rate (e.g., cooperative Hill kinetics). FIG. 10C depicts a relative amplification switch design, in which a reference miRNA (sequence Z) will bind to a second template (Z′rYptₘ′) to produce a decoy molecule (Yptₘ). The mathematical model is identical to that shown in FIG. 10B.

The templates disclosed herein (the transduction templates and DNA templates) can be included in a composition for the amplification and/or detection/identification of target nucleic acid molecules in a sample. Further, the templates disclosed herein (the transduction templates and DNA templates) can be included in kits for the amplification and/or detection/identification of target nucleic acid molecules in a sample. Kits of the present invention may comprise, for example, one or more polymerases, transduction and DNA templates, and one or more nicking enzymes, as described herein. Where one target is to be amplified, one or two nicking enzymes may be included in the kit. Where multiple target sequences are to be amplified, and the templates designed for those target sequences comprise the nicking enzyme sites for the same nicking enzyme, then one or two nicking enzymes may be included. Or, where the templates are recognized by different nicking enzymes, two or more nicking enzymes may be included in the kit, such as, for example, 3 or more.

The kits of the present invention may also comprise one or more of the components in any number of separate containers, packets, tubes, vials, microtiter plates and the like, or the components may be combined in various combinations in such containers.

The components of the kit may, for example, be present in one or more containers, for example, all of the components may be in one container, or, for example, the enzymes may be in one or more separate containers from the templates. The components may, for example, be lyophilized, freeze dried, or in a stable buffer. In one example, the polymerase and nicking enzymes are in lyophilized form in a single container, and the templates are either lyophilized, freeze dried, or in buffer, in a different container. Or, in another example, the polymerase, nicking enzymes, and the templates are, in lyophilized form, in a single container. Or, the polymerase and the nicking enzyme may be separated into different containers.

Kits may further comprise, for example, dNTPs used in the reaction, or modified nucleotides, cuvettes or other containers used for the reaction, or a vial of water or buffer for re-hydrating lyophilized components. The buffer used may, for example, be appropriate for both polymerase and nicking enzyme activity.

The kits of the present invention may also comprise instructions for performing one or more methods described herein and/or a description of one or more compositions or reagents described herein. Instructions and/or descriptions may be in printed form and may be included in a kit insert. A kit also may include a written description of an Internet location that provides such instructions or descriptions.

Kits may further comprise reagents used for detection methods, such as, for example, reagents used for FRET, lateral flow devices, dipsticks, fluorescent dye, colloidal gold particles, latex particles, a molecular beacon, or polystyrene beads.

### Aspects

A variety of aspects of methods, systems, compositions, and kits have been described herein. A summary of a few select examples of such methods, systems, compositions, and kits are provided below.

In a 1^{st} aspect, a method of detecting a target oligonucleotide sequence (X) is provided, said method comprising:

forming a reaction mixture that comprises:
(1) a target nucleic acid comprising a target oligonucleotide sequence (X);
(2) a first antisense template (X′R1t′Yp);
   wherein the first antisense template (X′R1t′Yp) comprises from 3′ to 5′: (a) a first sequence of nucleotides (X′) that is at least substantially complementary to the target oligonucleotide sequence (X); (b) a second sequence of nucleotides (R1) of an anti-sense strand of a first nicking enzyme binding site; and (c) a third sequence of nucleotides (t′Yp) that is at least substantially complementary to a reporter oligonucleotide sequence (tYp), wherein the third sequence of nucleotides (t′Yp) comprises from 3′ to 5′: (i) a toehold nucleotide sequence (t′); and (ii) a palindromic nucleotide sequence (Yp);
(3) a second antisense template (t′YpR2t′Yp);
   wherein the second antisense template (t′YpR2t′Yp) comprises from 3′ to 5′: (a) a fourth sequence of nucleotides comprising t′Yp; (b) a fifth sequence of nucleotides (R2) of an anti-sense strand of a second nicking enzyme binding site; and (c) a sixth sequence of nucleotides comprising t′Yp, wherein the two palindromic nucleotide sequences (Yp) of the second antisense template (t′YpR2t′Yp) cause the second antisense template (t′YpR2t′Yp) to form a palindrome and fold into a stem and loop configuration. (4) a polymerase;
(5) a first nicking enzyme that nicks at the first nicking enzyme binding site;
(6) a second nicking enzyme that nicks at the second nicking enzyme binding site; and
(7) nucleotides.
   subjecting the reaction mixture to essentially isothermal conditions at a reaction temperature to amplify the reporter oligonucleotide sequence (tYp) at a non-linear amplification rate;
   and detecting the reporter oligonucleotide sequence (tYp).

A 2^{nd} aspect is a method of the 1^{st} aspect, wherein the non-linear amplification rate of the reporter oligonucleotide sequence (tYp) demonstrates cooperative Hill kinetics.

A 3^{rd} aspect is a method of the 1^{st} or 2^{nd} aspect, the amplification of the reporter oligonucleotide is biphasic.

A 4^{th} aspect is a method of the 3^{rd} aspect, wherein the first phase linearly amplifies the oligonucleotide sequence (tYp) and the second phase amplifies the reporter oligonucleotide sequence (tYp) at a non-linear amplification rate.

A 5^{th} aspect is a method of any of aspects 1-4, wherein said method can detect the target oligonucleotide sequence (X) at a concentration of ≤10 picomolar.

A 6^{th} aspect is a method of any of aspects 1-5, wherein the reporter oligonucleotide sequence (tYp) is linearly amplified from the steps including: (A) forming a duplex (D1) comprising the target oligonucleotide sequence (X) and the first antisense template (X′R1t′Yp); (B) extending, using the polymerase, the target oligonucleotide sequence (X) of the duplex (D1) along the first antisense template (X′R1t′Yp) to form an extended target oligonucleotide sequence comprising a sense sequence complementary to the first antisense template (X′R1t′Yp); (C) nicking, with the first nicking enzyme, at the first nicking enzyme binding site on the sense strand of the duplex (D1) to produce the reporter oligonucleotide sequence (tYp); and (D) repeating steps (B) and (C) to thereby linearly amplify the reporter oligonucleotide sequence (tYp).

A 7^{th} aspect is a method of any of aspects 1-6, wherein the reporter oligonucleotide (tYp) is non-linearly amplified from the steps including: (A) forming a duplex (D2) comprising the reporter oligonucleotide sequence (tYp) and the second antisense template (t′YpR2t′Yp), wherein binding of the reporter oligonucleotide sequence (tYp) to the toehold site (t′) unfolds the stem and loop configuration of the second antisense template (t′YpR2t′Yp); (B) extending, using the polymerase, the reporter oligonucleotide sequence (tYp) of the duplex (D2) along the second antisense template (t′YpR2t′Yp) to form an extended reporter oligonucleotide sequence comprising a sense sequence complementary to the second antisense template (t′YpR2t′Yp); (C) nicking, with the second nicking enzyme, at the second nicking enzyme binding site on the sense strand of the duplex (D2) to produce the reporter oligonucleotide sequence (tYp); and (D) repeating steps (B) and (C) to thereby non-linearly amplify the reporter oligonucleotide sequence (tYp).

An 8^{th} aspect is a method of any of aspects 1-7, wherein the non-linear amplification rate of the reporter oligonucleotide sequence (tYp) demonstrates cooperative Hill kinetics.

A 9^{th} aspect is a method of any of aspects 1-8, wherein the first nicking binding site and the first nicking site are identical.

A 10^{th} aspect is a method of any of aspects 1-9, wherein the first nicking site and the second nicking site are nicked by the same nicking enzyme.

An 11^{th} aspect is a method of any of aspects 1-10, wherein the first sequence of nucleotides (X′) is completely complementary to the target oligonucleotide sequence (X).

A 12^{th} aspect is a method of any of aspects 1-11, wherein the third sequence of nucleotides (t′Yp) is completely complementary to the reporter oligonucleotide sequence (tYp).

A 13^{th} aspect is a method of any of aspects 1-12, wherein the 3′ terminus of the first antisense template (X′R1t′YP) and the 3′ terminus of the second antisense template (t′YpR2t′YP) are blocked.

A 14^{th} aspect is a method of any of aspects 1-13, detecting the reporter oligonucleotide sequence (tYp) is performed at least partially by a technique selected from the group consisting of luminescence spectroscopy or spectrometry, fluorescence, fluorescence spectroscopy or spectrometry, mass spectrometry, liquid chromatography, fluorescence polarization, colorimetry, and electrophoresis.

A 15^{th} aspect is a method of any of aspects 1-14, wherein detecting reporter oligonucleotide sequence (tYp) comprises detecting amplification of the reporter oligonucleotide sequence (tYp).

A 16^{th} aspect is a method of aspect 15, wherein the step of detecting amplification of the reporter oligonucleotide sequence (tYp) is performed at least partially by a technique selected from the group consisting of luminescence spectroscopy or spectrometry, fluorescence, fluorescence spectroscopy or spectrometry, mass spectrometry, liquid chromatography, fluorescence polarization, colorimetry, and electrophoresis.

A 17^{th} aspects is a method of any of aspects 1-16, wherein detecting reporter oligonucleotide sequence (tYp) comprises detecting an amplification rate of the reporter oligonucleotide sequence (tYp).

An 18^{th} aspect is a method of aspect 17, wherein the step of detecting the amplification rate of the reporter oligonucleotide sequence (tYp) is performed at least partially by a technique selected from the group consisting of luminescence spectroscopy or spectrometry, fluorescence, fluorescence spectroscopy or spectrometry, mass spectrometry, liquid chromatography, fluorescence polarization, colorimetry, and electrophoresis.

A 19^{th} aspect is a method of any of aspects 1-18, wherein the target nucleic acid comprising a target oligonucleotide sequence (X) is obtained from a sample derived from animal.

A 20^{th} aspect is a method of aspect 19, wherein the sample is blood, serum, mucus, saliva, urine, or feces.

A 21^{st} aspect is a method of any of aspects 1-19, wherein the target nucleic acid comprising a target oligonucleotide sequence (X) is any synthetic or natural RNA molecule, including mRNA, microRNA, and siRNA.

A 22^{nd} aspect is a method of any of aspects 1-19, wherein the target nucleic acid comprising a target oligonucleotide sequence (X) is any synthetic or natural DNA molecule, including genomic DNA, mitochondrial DNA, cDNA derived from reverse transcription of mRNA, microRNA, or siRNA.

A 23^{rd} aspect is a method of any of aspects 1-22, wherein said method further comprises a step of denaturing said target nucleic acid comprising a target oligonucleotide sequence (X) prior to forming the reaction mixture.

A 24^{th} aspect is a method of any of aspects 1-23, wherein the polymerase is a warm start polymerase.

A 25^{th} aspect is a method of any of aspects 1-24, wherein the amplification of the reporter oligonucleotide sequence (tYp) is performed at about 55° C. to about 60° C.

A 26^{th} aspect is a method of any of aspects 1-25, wherein the reporter oligonucleotide sequence (tYp) is from 8-30 nucleotides in length.

A 27^{th} aspect is a method of any of aspects 1-26, wherein the toehold site (t′) of the first, third, fourth, and fifth sequence of nucleotides is from 3-8 nucleotides in length.

A 28^{th} aspect is a method of any of aspects 1-27, wherein the palindrome of the second antisense template (t′YpR2t′Yp) is from 4-22 nucleotides in length.

A 29^{th} aspect is a method of any of aspects 1-28, wherein the palindrome of the second antisense template (t′YpR2t′Yp) has a melting temperature that is greater than the reaction temperature, but less than 90° C.

A 30^{th} aspect is a method of any of aspects 1-29, wherein the antisense template (t′YpR2t′YP) has a melting temperature that is greater than the reaction temperature, but less than 89° C., 88° C., 87° C., 86° C., 85° C., 84° C., 83° C., 82° C., 81° C., or 80° C.

A 31^{st} aspect is a method of any of aspects 1-30, wherein the duplex (D2) has a melting temperature that is less than the reaction temperature plus 5° C.

A 32^{nd} aspect is a method of any of aspects 1-31, wherein the nicking enzyme is selected from the group consisting of Nt.BstNBI, Nt.BspQI, Nb.BBvCl, Nb.Bsml, Nb.BsrDI, Nb.BstI, Nt.Alwl, Nt.BbvCI, Nt.CviPII, Nt.BsmAI, Nb.Bpu1oI, and Nt.Bpu10I.

### EXAMPLES

The following examples are given by way of illustration and are in no way intended to limit the scope of the present invention.

### Example 1

### Materials and Methods

Unless specified otherwise, the following experimental techniques were used in the Examples.

### Reagents:

UltraPure™ Tris-HCl pH 8.0, RNase free EDTA, RNase free MgCl2, RNase free KCl, Novex™ TBE Running Buffer (5×), 2×TBE-Urea Sample Buffer, Novex™ TBE-Urea Gels, 15%, SYBR® Gold Nucleic Acid Gel Stain, and SYBR® Green II RNA Gel Stain were purchased from Thermo Fisher Scientific (Waltham, Mass.). Nuclease-free water and oligo length standard 10/60 were purchased from Integrated DNA Technologies, Inc. (Coralville, Iowa). Nt.BstNBI nicking endonuclease, Bst 2.0 WarmStart® DNA Polymerase, 10× ThermoPol I Buffer, dNTPs, BSA, and 100 mM MgSO4 were purchased from New England Biolabs (Beverly, Mass.).

Oligonucleotides were ordered from two different sources to avoid trigger contamination in templates. Desalted amplification templates were purchased from Integrated DNA Technologies (Coralville, Iowa) suspended in IDTE Buffer at a concentration of 100 μM. Templates were modified with an amino group on the 3′ end to prevent template extension. All desalted trigger oligonucleotides were purchased from Eurofins Genomics (Louisville, Ky.) suspended at a concentration of 50 μM in TE Buffer. Triggers were diluted in nuclease-free water in a separate room to prevent contamination.

### Template Design and Thermodynamics:

Thermodynamics of the template stem loops were determined using the Mfold web server31, an open source software that uses empirical free energies of DNA hybridization32that have been corrected for salt concentration33 (http://unafold.rna.albany.edu/?q=mfold). The free energies of association between the template and trigger, template and elongated trigger, product dimers, and double stranded templates were determined using the DINAmelt application, two-state melting (http://unafold.rna.albany.edu/?q=DINAMelt/Two-state-melting). To determine the free energy of toehold association, the software input was the sequence of the toehold and the toehold reverse compliment. All settings used were kept at the default software parameters, except for temperature (55° C.) and salt concentration ([Na+]=60 mM, [Mg++]=6 mM).

### Biphasic Amplification Reactions:

The amplification reaction mixture contained 1× ThermoPol I Buffer [20 mM Tris-HCl (pH 8.8), 10 mM (NH₄)₂SO₄, 10 mM KCl, 2 mM MgSO₄, 0.1% Triton® X-100], 25 mM Tris-HCl (pH 8), 6 mM MgSO₄, 50 mM KCl, 0.5 mM each dNTP, 0.1 mg/mL BSA, 0.2 U/μL Nt.BstNBI, and 0.0267 U/μL Bst 2.0 WarmStart® DNA Polymerase. Bst 2.0 WarmStart® DNA polymerase is inactive below 45° C.; this decreases non-specific amplification before reaction initiation and theoretically increases experimental reproducibility. Templates were diluted in nuclease-free water and added at a final concentration of 100 nM. SYBR Green II (10,000× stock in DMSO) was added to the reaction mixture to a final concentration of 5×. Reactions were prepared at 4° C., and triggers and templates were handled in separate hoods to prevent contamination. Triggers were diluted in nuclease-free water and added to positive samples to a final concentration of 10 pM unless otherwise indicated; negative controls contained no trigger. For each experiment, two controls were prepared: a no-template control (NTC) sample containing no template, and a no-enzyme control sample containing no enzymes. Reactions were run in triplicate 20 μL volumes. Fluorescence readings were measured using a Bio-Rad CFX Connect Thermocycler (Hercules, Calif.). Measurements were taken every 20 seconds with a 12 second imaging step. Reactions were run for either 150 or 300 cycles of 32 seconds at 55° C. The mixture was heated to 80° C. for 20 minutes to deactivate enzymes, followed by 10° C. for five minutes to cool the samples. Completed reactions were stored at −20° C. for further analysis.

### Product Quantification:

NanoDrop 3300 Fluorospectrometer (Thermo Scientific, Wilmington, Del.) was used for measuring the reaction product concentrations. The standards (ssDNA oligos, Eurofins Genomics, Louisville, Ky.) and the reaction products were diluted in 1×TE buffer (1 mM Tris-HCl, 0.5 mM EDTA) if needed. Nucleic acid stains were diluted in 1×TE Buffer. 1×SYBR® Gold Nucleic Acid Gel Stain (for low concentration samples) or 2.5×SYBR® Green II RNA Gel Stain (for high concentration samples), and 1.2 μL of the sample were brought to a final volume of 12 μL with 1×TE buffer. The standards were prepared in the same way as the reaction products with the addition of mock reaction product (reaction components without enzymes or trigger) and triggers diluted in 1× TE Buffer. Samples were excited with blue light (470±10 nm) with autogain on. The fluorescence peaks of the dyes were determined to be 512 nm for SYBR® Green II RNA Gel Stain and 536 nm for SYBR® Gold Nucleic Acid Gel Stain for the specific salt conditions, and the average fluorescence of 5 replicate measurements was used to determine the product concentrations of the reaction products. 1×TE buffer was used as the blank measurement.

### Data Analysis:

Real-time reaction traces were analyzed with custom software using Matlab (Natick, Mass.). Inflection points were defined as the peaks in the first derivative of the fluorescence with respect to time. To determine exact inflection points, the top of the peak was fit to a quadratic function dF/dt=at2+bt+c where F is fluorescence and t is time. The inflection point was defined as the zero of the second derivative (tIF=2at+b). The first plateau was defined as the fluorescence corresponding to the time of the lowest point between the first and second peaks in the first derivative. The maximum reaction rates were defined as the top of the peaks in the first derivative (maximum dF/dt). The ratios between maximum reaction rates, plateaus, and inflection points were calculated from two experiments with three experimental replicates each. When appropriate, data from two experiments were averaged using a weighted average. Spearman's rank-order correlations and p-values were determined using the function “con” with the type selected as “Spearman” in Matlab (Natick, Mass.). Weighted averages (x_{wav}) from average experimental triplicates (xᵢ) standard deviations (σᵢ) were calculated when appropriate by Eq. 2 as follows:

$${x}_{wav} = \frac{\sum {w}_{i} {x}_{i}}{\sum {w}_{i}} , {w}_{i} = \frac{1}{{σ}_{i}^{2}} , and {σ}_{wav}^{2} = \frac{1}{\sum {w}_{i}} . \qquad Eq . 2$$

Standard curves relating to fluorescence to trigger DNA concentration were fit to a simple linear regression model using statistical software RStudio. The mean trigger concentration predictions of the unknowns were obtained using the “inverse.predict” function of RStudio, under the package “chemCal.” Standard deviations were computed using the function “stdev” in the electronic spreadsheet program Microsoft Excel. Standard error from the predicted concentration was converted to standard deviation and the cumulative standard deviation of the trigger DNA concentration was calculated using Eq. 3 as follows:

$${SD}_{total} = \sqrt{{SD}_{prediction}^{2} + {SD}_{sample mean of the replicates}^{2}} . \qquad Eq . 3$$

### Results and Discussion

### Reaction Pathways in the Biphasic DNA Amplification Reaction:

The biphasic DNA amplification reaction contains the same basic components as the exponential amplification reaction for oligonucleotides (EXPAR). Both EXPAR and the biphasic DNA amplification reaction amplify a trigger sequence at a substantially single reaction temperature (e.g., 55° C.) through the action of a thermophilic polymerase and a nicking endonuclease. The main difference between the original EXPAR reaction and the biphasic oligonucleotide amplification reaction is the palindromic sequence within the DNA template that causes the template to fold into a looped configuration. The thermodynamics of the trigger binding and DNA template association are in a regime that creates a biphasic DNA amplification re-action.

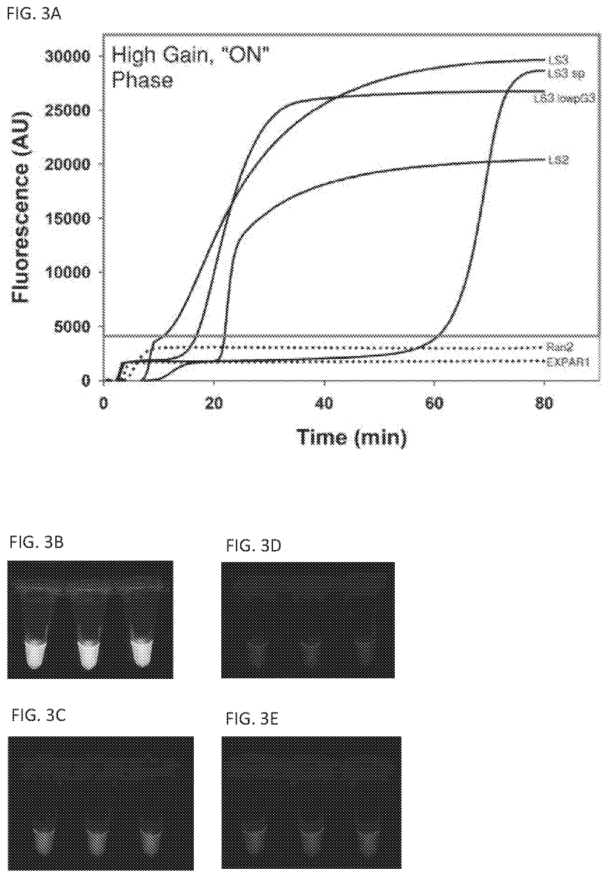

Representative outputs of the oligonucleotide amplification reaction are shown in FIGS. 3A-E. DNA template names (second antisense template (t′YpR2t′Yp)), as well as their associated triggers (reporter oligonucleotide sequence (tYp)) can be found in Table 1. Referring to Table 1, bases in italics include the toehold, bases in bold are the palindromic sequences that form the palindrome, and the underlined bases show the enzyme binding site. A nucleotide that is both bold and italic represents a base in the palindrome that is not predicted to be bound by Mfold web server, an open source software that uses empirical free energies of DNA hybridization that have been corrected for salt concentration (http://unafold.rna.albany.edu/?q=mfold), making it both a part of the palindromic sequence and part of the toehold. Despite the similarities in reaction components, the biphasic amplification reaction reported here is functionally distinct from all other EXPAR reactions. The first phase of the reaction resembles traditional EXPAR output, with an initial rise and a first plateau. Thermodynamics of the looped DNA template and trigger association are well correlated with the first-phase reaction kinetics (Spearman's R=0.8022, data not shown.) when compared to the original EXPAR reaction (R=0.4072). This is likely due to the closed template loop; thermodynamics of DNA association dominate the reaction kinetics, contrasting the sequence dependence seen in traditional EXPAR. After the first plateau, the biphasic reaction enters a high-gain second phase. This finding reveals that EXPAR can recover from the first plateau, a fact that was previously unknown. The one template that favors a linear configuration at the reaction temperature (LS3 lowpG2, Tm=49.2 □C) gives biphasic output, implying that while a palindromic region is necessary for biphasic output, a stable loop structure is not.

TABLE 1
Template and trigger sequences.

| Template | | |
|---|---|---|
| Name | Sequence | Trigger |
| LS2 | 5′-TCCGGAGAATTAATGACTCTTCCGG [symbol] GAAT-3′ NH₂ | 5′-ATTCTCCGGA-3′ |
| | (SEQ ID NO: 1) | (SEQ ID NO: 37) |
| LS3 | 5′-CGCGCGGTTTGGTAATGACTCTCGCGCGGTTTGG-3′ NH₂ | 5′-CAAACCGCGCG-3′ |
| | (SEQ ID NO: 2) | (SEQ ID NO: 38) |
| LS2-1 | 5′-ACCGGTGAATTAATGACTCTACCGGTGAAT-3′ NH₂ | 5′-ATTCACCGGT-3′ |
| | (SEQ ID NO: 3) | (SEQ ID NO: 39) |
| LS2-2 | 5′-AGCGCTGAATTAATGACTCTAGCGCTGAAT-3′ NH₂ | 5′-ATTCAGCGCT-3′ |
| | (SEQ ID NO: 4) | (SEQ ID NO: 40) |
| LS2-3 | 5′-TGCGCAGAATTAATGACTCTTGCGCAGAAT-3′ NH₂ | 5′-ATTCTGCGCA-3′ |
| | (SEQ ID NO: 5) | (SEQ ID NO: 41) |
| LS2-4 | 5′-TCCGGAGAATAAAAGACTCTTCCGGAGAAT-3′ NH₂ | 5′-ATTCTCCGGA-3′ |
| | (SEQ ID NO: 6) | (SEQ ID NO: 37) |
| LS2-5 | 5′-TCCGGAGAATAAAGGACTCTTCCGGAGAAT-3′ NH₂ | 5′-ATTCTCCGGA-3′ |
| | (SEQ ID NO: 7) | (SEQ ID NO: 37) |
| LS2-6 | 5′-TGGCCAGAATTAATGACTCTTGGCCAGAAT-3′ NH₂ | 5′-ATTCTGGCCA-3′ |
| | (SEQ ID NO: 8) | (SEQ ID NO: 42) |
| LS2-7 | 5′-TCGCGAGAATTAATGACTCTTCGCGAGAAT-3′ NH₂ | 5′-ATTCTCGCGA-3′ |
| | (SEQ ID NO: 9) | (SEQ ID NO: 43) |
| LS2-8 | 5′-TCCGGACAATTAATGACTCTTCCGGACAAT-3′ NH₂ | 5′-ATTGTCCGGA-3′ |
| | (SEQ ID NO: 10) | (SEQ ID NO: 44) |

Long random sequence in the loop-increase the loop
opening AG, Decrease the elongated trigger:template AG

| | | |
|---|---|---|
| LS2lrs-1 | 5′-TCCGGAGAATTAATGACTCTGCTTTCCGG [symbol] GAAT-3′ NH₂ | 5′-ATTCTCCGGA-3′ |
| | (SEQ ID NO: 11) | (SEQ ID NO: 37) |
| LS2lrs-4 | 5′-TCCGGAGAATTAATGACTCTGCTTAGTCAGTCCGG [symbol] GAAT-3′ NH₂ | 5′-ATTCTCCGGA-3′ |
| | (SEQ ID NO: 12) | (SEQ ID NO: 37) |
| LS3lrs-2 | 5′-CGCGCGGTTTGGTAATGACTCTGCTTAGCGCGCGGTTTGG-3′ NH₂ | 5′-CCAAACCGCGCG-3′ |
| | (SEQ ID NO: 13) | (SEQ ID NO: 45) |
| LS3lrs-4 | 5′-CGCGCGTTTGGTAATGACTCTACTTGGCTTACGCGCGGTTGG-3′ NH₂ | 5′-CCAAACCGCGCG-3′ |
| | (SEQ ID NO: 14) | (SEQ ID NO: 45) |
| LS52lowtGlrs1 | 5′-TCCGGATAATTAATGACTCTGCTTTCCGG [symbol] TAAT-3′ NH₂ | 5′-ATTATCCGGA-3′ |
| | (SEQ ID NO: 15) | (SEQ ID NO: 46) |
| LS2lowtglrs2 | 5′-TCCGGATAATTAATGACTGCTGCTTTCCGG [symbol] TAAT-3′ NH₂ | 5′-CCAAACTAGCTA-3′ |
| | (SEQ ID NO: 16) | (SEQ ID NO: 47) |
| LS3lt-1lrs1 | 5′-CGCGCGGTTTGGACGTAATGACTCTGCTTCGCGCGGTTTGGACG-3′ NH₂ | 5′-CGTCCAAACCGCGCG-3′ |
| | (SEQ ID NO: 17) | (SEQ ID NO: 48) |
| LS3lt-1rs2 | 5′-CGCGCGGTTTGGACGTAATGACTCTGCTTAGTCAGCGCGCGGTTTGGACG-3′ NH₂ | 5′-CGTCCAAACCGCGCG-3′ |
| | (SEQ ID NO: 18) | (SEQ ID NO: 48) |

Vary palindrome length and GC content-change
palindrome AG and elongated trigger:template AG

| | | |
|---|---|---|
| LS3lp*++ | 5′-CGCGCGCGGTTTGTAATGACTCTCGCGCGCGGTTG-3′ NH₂ | 5′-CAAACCGCGCGCG-3′ |
| | (SEQ ID NO: 19) | (SEQ ID NO: 99) |
| LS3lowpG3 | 5′-TCCGGAGTTTGGTAATGACTCTTCCGG [symbol] GTTTGG-3′ NH₂ | 5′-CCAAACTCCGGA-3′ |
| | (SEQ ID NO: 20) | (SEQ ID NO: 50) |
| LS3 sp | 5′-CGCGGTTTATTAATGACTCTCGCGGTTTAT-3′ NH₂ | 5′-CCAAACCGCG-3′ |
| | (SEQ ID NO: 21) | (SEQ ID NO: 51) |
| LS3lowpG2 | 5′-TAGCTAGTTTGGTAATGACTCTTAGCTAGTTTGG-3′ NH₂ | 5′-CCAAACCGCG-3′ |
| | (SEQ ID NO: 22) | (SEQ ID NO: 51) |
| LS2hpG1*++ | 5′-GCCGGCGAATTAATGACTCTGCCGGCGAAT-3′ NH₂ | 5′-ATTCGCCGGC-3′ |
| | (SEQ ID NO: 23) | (SEQ ID NO: 52) |
| LS2lp*++ | 5′-GCCGGCGAATTAATGACTCTGCCGGCGAAT-3′ NH₂ | 5′-ATTCGCCGGC-3′ |
| | (SEQ ID NO: 24) | (SEQ ID NO: 52) |
| LS2 sp+ | 5′-CCGGGAATTAATGACTCTCCGGGAAT-3′ NH₂ | 5′-ATTCCCGG-3′ |
| | (SEQ ID NO: 25) | (SEQ ID NO: 53) |

Vary toehold length and GC content-change
toehold AG and elongated trigger; template AG

| | | |
|---|---|---|
| LS3 lt1 | 5′-CGCGCGGTTTGGACGTAATGACTCTCGCGCGGTTTGGACG-3′ NH₂ | 5′-CGTCCAAACCGCGCG-3′ |
| | (SEQ ID NO: 26) | (SEQ ID NO: 48) |
| LS3 htG++ | 5′-CGCGCGGTGCGGTAATGACTCTCGCGCGGTGCGG-3′ NH₂ | 5′-CCGCACCGCGCG-3′ |
| | (SEQ ID NO: 27) | (SEQ ID NO: 54) |
| LS3 lowtG | 5′-CGCGCGGTTTATTAATGACTCTCGCGCGGTTTAT-3 NH₂ | 5′-ATAAACCGCGCG-3′ |
| | (SEQ ID NO: 28) | (SEQ ID NO: 55) |
| LS2 htG2 | 5′-TCCGGAGCGCTAATGACTCTTCCGG [symbol] GCGC-3′ NH₂ | 5′-GCGCTCCGGA-3′ |
| | (SEQ ID NO: 29) | (SEQ ID NO: 56) |
| LS2 lt3 | 5′-TCCGGAGAATGATCTAATGACTCTTCCGG [symbol] GAATGATC-3′ NH₂ | 5′-GATCATTCTCCGGA-3′ |
| | (SEQ ID NO: 30) | (SEQ ID NO: 57) |
| LS2 s‡ | 5′-TCCGGAGATAATGACTCTTCCGGAGA-3′ NH₂ | 5′-TCTCCGGA-3′ |
| | (SEQ ID NO: 31) | (SEQ ID NO: 58) |
| LS2 lowtG | 5′-TCCGGATAATTAATGACTCTTCCGG [symbol] TAAT-3′ NH₂ | 5′-ATTATCCGGA-3′ |
| | (SEQ ID NO: 32) | (SEQ ID NO: 59) |

No 5′ toehold-lock the 5′ toehold using added nucleotides in the loop-
decrease loop AG and elongated trigger:template AG, remove cooperative binding of trigger

| | | |
|---|---|---|
| LS2no5*++ | 5′-TCCGGAGAATTAATGACTCTATTCTCCGG [symbol] GAAT-3′ NH₂ | 5′-ATTCTCCGGA-3′ |
| | (SEQ ID NO: 33) | (SEQ ID NO: 37) |
| LS2no5′lrs3*++ | 5′-TCCGGAGAATTAATGACTCTGTATAGCTATTCTCCGG [symbol] GAAT-3′ NH₂ | 5′-ATTCTCCGGA-3′ |
| | (SEQ ID NO: 34) | (SEQ ID NO: 37) |

Linear templates

| | | |
|---|---|---|
| Ran2**+ | 5′-GGGGAAATAGGTGAGACTCTGGGGAAATAG-3′ NH₂ | 5′-CTATTTCCCC-3′ |
| | (SEQ ID NO: 35) | (SEQ ID NO: 60) |
| EXPAR1**+ | 5-′CTCACGCTACGGACGACTCTCTCACGCTAC-3′ PO₃ | 5′-GTAGCGTGAG-3′ |
| | (SEQ ID NO: 36) | (SEQ ID NO: 61) |

+= did not enter the second phase;
++= did not have a measurable first phase;
*= ab initio synthesis after approximately 80 mm;
**= ab initio synthesis after approximately 30 mm;
‡= no amplification.

The detailed mechanism behind the switch-like oligonucleotide amplification reaction is likely driven by multiple phenomena, as shown in FIG. 2A and previously discussed. Without being bound by theory, these new reaction pathways create the unique features of our amplification reaction as detailed in FIG. 2B and also previously discussed.

### Properties of the First Reaction Phase:

The first reaction phase resembles the base EXPAR reaction, with a rapid, low-gain reaction phase followed by a plateau. While this stall was previously attributed to loss of nickase integrity, the recovery of the reaction after the first plateau invalidates this theory. Recently others have hypothesized that some templates could be “poisoned” due to polymerase errors that render the DNA strand bound to the template unextendible (FIG. 2A, subpanel 5). Without being bound by theory, this could cause the plateau seen in the original EXPAR reaction and the first plateau in the biphasic amplification reaction (FIG. 2B). We estimated the plateau trigger concentration to be on the order of 1 μM (data not shown), which is ten times greater than the template concentration. Due to the rapid template inactivation after ten cycles of ex-tension and nicking it is unlikely that polymerase error causes this plateau, given error rates of polymerases such as Bst DNA polymerase that lack the 3′→5′ exonuclease domain are approximately 10⁻⁴⁴². Without being bound by theory, this plateau may be due to noncanonical behavior of the nickase enzyme that leaves a long unextendible trigger (FIG. 2A, subpanel 4), as the nickase is operating in suboptimal conditions when compared to the polymerase. It is also possible that a fully elongated trigger poisons the template; the mechanism behind the template poisoning are not addressed herein.

### Properties of the Second Reaction Phase:

After the first plateau, the amplification enters a high-gain second phase followed by a second plateau. The amplification does not exit the first plateau unless there is a palindromic region in the template; we hypothesize that template rescue is aided by trigger association to the long “poisoned” triggers (FIG. 2A, subpanel 4). This trigger-dependent rescue would prevent the long trigger from reassociating with the template, particularly after polymerase extension of the 3′ trigger end. Without being bound by theory, the trigger could also dynamically bind the template and prevent reassociation of the long trigger. These events would aid in the loop closure and template rescue. After exiting the first plateau many of the templates exhibit Hill-like second phase kinetics, marked by a large jump in reaction product that greatly exceeds first phase reaction kinetics. Without being bound by theory, this ultra-sensitivity may be caused by homotropic allosteric cooperativity; the trigger can bind either toehold as seen in FIG. 2A, subpanel 1. The template loop structure is stable when compared to the trigger:template association (Table 2), and the accumulation of reaction products would shift templates to an open, amplification competent state and produce nonlinear reaction kinetics (d([trigger])/dt∝[trigger]²). Table 2 shows the template of the looped template structures. All templates contained two open toeholds and a palindromic loop, with the exception of the no 5′ toehold templates. Thermodynamics are also given for trigger:template association and long trigger:template association, where the long trigger is the original trigger with the elongated recognition site.

TABLE 2
Template Thermodynamics:

| | 3′ toe ΔG, | 5′ toe ΔG, | Palindrome | Palindrome | Loop ΔG, | |
|---|---|---|---|---|---|---|
| Template Name | kcal/mol | kcal/mol | ΔG, kcal/mol | Tₑₛ | kcal/mol | Loop Tₑₛ |
| LS2 | −0.4 | 0.2 | −3.9 | 27.0° C. | −1.6 | 67.1° C. |
| LS3 | −2.0 | −2.0 | −5.4 | 42.2° C. | −3.66 | 76.7° C. |

Long random sequence in the loop - Increase the loop opening ΔG, Decrease the elongated
trigger:template ΔG

| | | | | | | |
|---|---|---|---|---|---|---|
| LS2 lrs-1 | −0.4 | 0.2 | −3.9 | 27.0° C. | −1.14 | 63.7° C. |
| LS2 lrs-4 | −0.4 | 0.2 | −3.9 | 27.0° C. | −0.49 | 58.8° C. |
| LS3 lrs-2 | −2.0 | −2.0 | −5.4 | 42.2° C. | −3.25 | 73.5° C. |
| LS3 lrs-4 | −2.0 | −2.0 | −5.4 | 42.2° C. | −2.8 | 71.2° C. |
| LS2 lowtG lrs1 | 0.4 | 0.4 | −3.9 | 27.0° C. | −1.06 | 63.2° C. |
| LS2 lowtG lrs2 | 0.4 | 0.4 | −3.9 | 27.0° C. | −0.26 | 56.9° C. |
| LS3 lt-1 lrs2 | −5.2 | −5.2 | −5.4 | 42.2° C. | −3.03 | 72.7° C. |
| LS3 lt-1 lrs2 | −5.2 | −5.2 | −5.4 | 42.2° C. | −2.41 | 68.5° C. |

Very Palindrome length and GC content - change palindrome ΔG and elongated
trigger:template ΔG

| | | | | | | |
|---|---|---|---|---|---|---|
| LS3 lp*†† | −0.7 | −0.7 | −8.6 | 60.3° C. | −6.97 | 86.5° C. |
| LS3 lowpG3 | −2.8 | −2.0 | −3.9 | 27.0° C. | −1.35 | 65.4° C. |
| LS3 sp | −2.0 | −2.0 | −2.2 | 7.0° C. | −0.46 | 58.9° C. |
| LS3 lowpG2 | −2.8 | −2.0 | −2.2 | 12.7° C. | 0.73 | 49.2° C. |
| LS2 hpG1*†† | 0.2 | 0.2 | −5 | 38.3° C. | −3.56 | 78.9° C. |
| LS2 lp*†† | 0.2 | 0.2 | −5 | 41.9° C. | −3.56 | 73.7° C. |
| LS2 sp† | 0.2 | 0.2 | −1.7 | −4.7° C. | −0.15 | 56.4° C. |

Very toehold length and content - change toehold ΔG and elongated trigger:template ΔG

| | | | | | | |
|---|---|---|---|---|---|---|
| LS3 lt1 | −5.2 | −5.2 | −5.4 | 42.2° C. | −3.45 | 75.3° C. |
| LS3 htG†† | −4.3 | −4.3 | −5.4 | 42.2° C. | −3.66 | 76.7° C. |
| LS3 lowtG | −0.5 | −0.5 | −5.4 | 42.2° C. | −3.66 | 76.7° C. |
| LS2 htG2 | −3.6 | −2.4 | −3.9 | 27.0° C. | −1.56 | 67.1° C. |
| LS2 lt3 | −3.4 | −2.8 | −3.9 | 27.0° C. | −1.14 | 63.7° C. |
| LS2 s†‡ | 0.1 | 1 | −3.9 | 27.0° C. | −1.67 | 68.0° C. |
| LS2 lowtG | 0.4 | 0.4 | −3.9 | 27.0° C. | −1.46 | 66.6° C. |

No 5′ toehold-lock the 5′ toehold using added nucleotides in the loop - decrease loop ΔG
end elongated trigger:template ΔG, remove cooperative binding of trigger

| | | | | | | |
|---|---|---|---|---|---|---|
| LS2 no5′*†† | −0.4 | n/a | −3.9 | 27.0° C. | −4.05 | 73.9° C. |
| LS2 no5′lrs3*†† | −0.4 | n/a | −3.9 | 27.0° C. | −3.31 | 70.3° C. |

Linear templates

| | | | | | | |
|---|---|---|---|---|---|---|
| Ran2**† | n/a | n/a | n/a | n/a | n/a | n/a |
| EXPAR1**† | n/a | n/a | n/a | n/a | n/a | n/a |

| | | | Elongated | |
|---|---|---|---|---|
| | Trigger:Template | | Trigger:Template | Elongated |
| | ΔG, | Trigger:Template | ΔG, | Trigger:Template |
| Template Name | kcal/mol | Tₑₛ | kcal/mol | Tₑₛ |
| LS2 | −6.9 | 47.5° C. | −13.0 | 65.5° C. |
| LS3 | −11.6 | 65.4° C. | −18.5 | 74.7° C. |

Long random sequence in the loop - Increase the loop opening ΔG, Decrease the elongated
trigger:template ΔG

| | | | | |
|---|---|---|---|---|
| LS2 lrs-1 | −6.9 | 47.5° C. | −16.8 | 70.5° C. |
| LS2 lrs-4 | −6.9 | 47.5° C. | −21.4 | 74.5 |
| LS3 lrs-2 | −11.7 | 65.6° C. | −24.0 | 79.1° C. |
| LS3 lrs-4 | −11.9 | 66.1° C. | −26.0 | 79.0° C. |
| LS2 lowtG lrs1 | −5.8 | 42.2° C. | −15.7 | 68.7° C. |
| LS2 lowtG lrs2 | −5.8 | 42.2° C. | −22.2 | 74.3° C. |
| LS3 lt-1 lrs2 | −14.8 | 71.8° C. | −25.5 | 80.3° C. |
| LS3 lt-1 lrs2 | −14.9 | 71.9° C. | −30.5 | 82.3° C. |

Very Palindrome length and GC content - change palindrome ΔG and elongated
trigger:template ΔG

| | | | | |
|---|---|---|---|---|
| LS3 lp*†† | −13.5 | 69.9° C. | −20.3 | 77.0° C. |
| LS3 lowpG3 | −8.9 | 56.7° C. | −15.4 | 69.2° C. |
| LS3 sp | −8.4 | 55.0° C. | −15.3 | 70.0° C. |
| LS3 lowpG2 | −6.8 | 49.3° C. | −13.0 | 64.3° C. |
| LS2 hpG1*†† | −9.1 | 58.1° C. | −16 | 71.9° C. |
| LS2 lp*†† | −9.1 | 57.6° C. | −16 | 70.5° C. |
| LS2 sp† | −5.6 | 38.3° C. | −12.4 | 65.1° C. |

Very toehold length and content - change toehold ΔG and elongated trigger:template ΔG

| | | | | |
|---|---|---|---|---|
| LS3 lt1 | −14.8 | 71.8° C. | −21.7 | 77.8° C. |
| LS3 htG†† | −13.9 | 72.4° C. | −20.8 | 78.9° C. |
| LS3 lowtG | −10.1 | 61.1° C. | −17 | 72.8° C. |
| LS2 htG2 | −9.8 | 60.6° C. | −16.2 | 72.0° C. |
| LS2 lt3 | −9.4 | 59.6° C. | −16 | 70.3° C. |
| LS2 s†‡ | −5.9 | 40.4° C. | −12.4 | 65.2° C. |
| LS2 lowtG | −5.6 | 42.2° C. | −11.9 | 63.0° C. |

No 5′ toehold-lock the 5′ toehold using added nucleotides in the loop - decrease loop ΔG
end elongated trigger:template ΔG, remove cooperative binding of trigger

| | | | | |
|---|---|---|---|---|
| LS2 no5′*†† | −6.9 | 47.5° C. | −15 | 67.4° C. |
| LS2 no5′lrs3*†† | −6.9 | 47.5° C. | −20.4 | 72.1° C. |

Linear templates

| | | | | |
|---|---|---|---|---|
| Ran2**† | −5.1 | 41.2° C. | −13.9 | 67.4° C. |
| EXPAR1**† | −6.5 | 47.4° C. | −16.7 | 73.3° C. |

†= did not enter second phase,
††= dit not have measureable first phase,
*= ob initio synthesis after approximately 80 min,
**= ob initio synthesis after approximately 30 min,
‡= no amplification

The subsequent second plateau is caused by exhaustion of reaction components and a buildup of inhibitory reaction by-products. This effect of inhibitory products was previously described when using EXPAR reactions and a palindromic looped template. The final output of the second phase is approximately the size of the DNA template triggers as seen in PAGE analysis of reaction products. This rescue of the poisoned templates allows the reaction to produce 10-100 times more endpoint reaction product as measured by calibrated SYBR II fluorescence. Endpoint product concentration ranged from 7.8-116.9 μM, with several reaction products exceeding 100 μM during the second plateau (Table 3). Table 3 shows the endpoint concentrations of trigger, quantified at 9632 seconds unless otherwise indicated. Samples were taken from the reaction endpoints and quantified using calibrated SYBR fluorescence. Errors represent standard deviation of the endpoint measurement.

TABLE 3
Endpoint concentrations of trigger:

| | | |
|---|---|---|
| | Estimated | |
| Template | [Product] (μM) | SD (μM) |

| | |
|---|---|
| LS3 lp* | 27.7 ± 4.2 |
| LS3 | 80.9 ± 4.6 |
| LS3 lrs-2 | 76.5 ± 4.5 |
| LS3 lrs-4 | 70.9 ± 4.5 |
| LS3 lt1 | 90.7 ± 5.0 |
| LS3 htG | 95.9 ± 4.9 |
| LS3 lowtG | 63.9 ± 4.2 |
| LS3 lt1 lrs-1 | 54.1 ± 4.2 |
| LS3 lt1 lrs-2 | 49.3 ± 4.1 |
| LS2 hpG1* | 5.3 ± 1.0 |
| LS2 lp* | 12.2 ± 1.0 |
| LS2 | 82.0 ± 7.5 |
| LS2 lrs-1 | 80.1 ± 7.5 |
| LS2 lrs-4 | 87.4 ± 8.7 |
| LS2 lt3 | 114.3 ± 7.8 |
| LS2 htG2 | 116.9 ± 9.5 |
| LS2 lowtG | 83.0 ± 7.5 |

*ab initio synthesis detected, quantified after 4800 s,
**ab initio synthesis detected, quantified after 1800 s.

### Reaction Response to Initial Trigger Concentration:

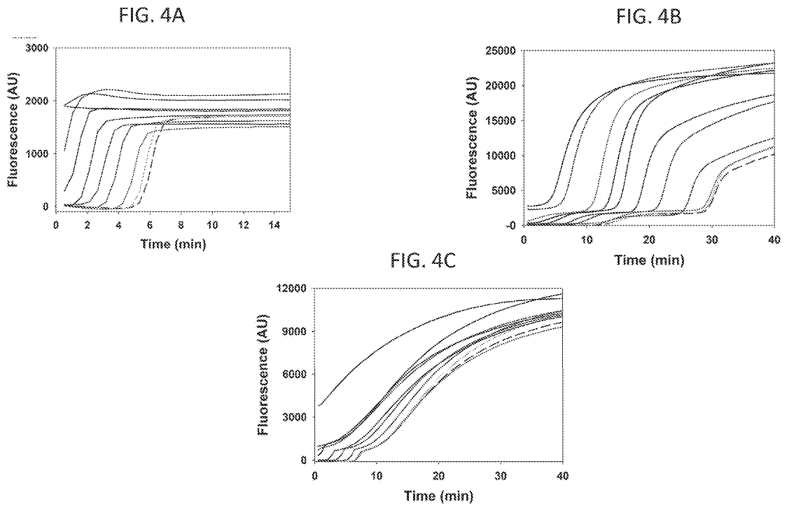

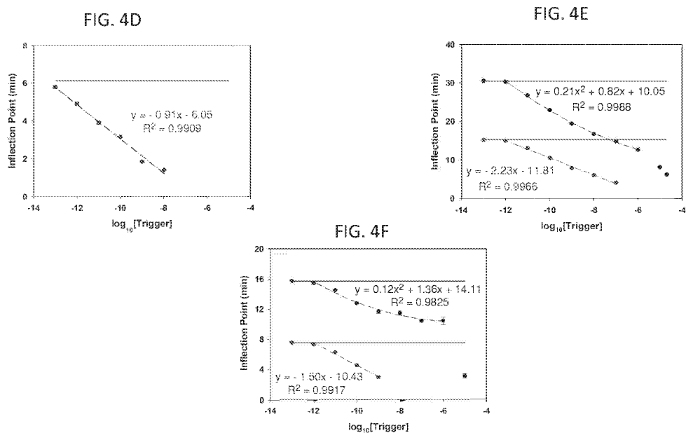

We measured reaction output in real-time with varying initial trigger concentrations using the ssDNA binding dye SYBR II for fluorescent readout. FIGS. 4A-C show representative real-time fluorescent traces. The average background fluorescence from samples with original trigger concentrations ≤10 nM was subtracted from all samples. It is important to note that absolute fluorescence units are arbitrary; indeed, the resolution between 1 μM and 10 μM fluorescence levels is smaller than the background noise between samples in FIG. 4A. This fluorescence variation does not a□ect the shape and inflection points of the graph, however. Traces are shown for three di□erent template types: a traditional EXPAR linear template (EXPAR1), a type I template (LS2 lowtG), and a type II template (LS3), respectively. The traditional EXPAR template was chosen from 384 published sequences as it had the highest separation between positive and negative controls. Fits were performed on inflection points for initial trigger concentrations between the lowest detectable concentration (100 fM or 1 pM) and the highest concentration below the first plateau (1 μM).

The calculated first and second inflection points are shown in FIGS. 4D-F. The traditional EXPAR template (FIG. 4A and FIG. 4D) did not enter the second phase, even when initial trigger concentration (10 μM) was above the plateau concentration of this template (5.58 μM, Table 3). Inflection points of the traditional template were linearly correlated with the log 10 of the original trigger concentration as expected. Inflection points in the first phase for both the type I and II looped templates were also linearly correlated with the log 10 of the original trigger concentration, but the correlation appeared slightly non-linear during the second phase (FIG. 4E and FIG. 4F). Type I templates have triggers that will dynamically dissociate from the template after nicking (trigger Tm<reaction temperature+5° C., so Tm<60° C. at a reaction temperature of 55° C.), while type II templates had stable trigger template associations that were more likely to remain until strand displacement by the polymerase (trigger Tm>reaction temperature+5° C., so Tm>60° C. at a reaction temperature of 55° C.). When the initial concentration of trigger exceeded the concentration at the first plateau, the inflection points appear to occur earlier than the fit lines predicted, which is expected for a hill-type reaction. The type I template initiated in the first plateau for initial trigger concentrations >1 μM and showed a short lag in amplification that was not present in the type II templates. The type I template also has a higher second plateau fluorescence level for greater initial concentrations of trigger, although it is unclear why this occurred. The type II template initiated in the second phase when the initial trigger concentration was 10 μM, which was above the measured plateau concentration (2.5±0.2 μM, data not shown). This demonstrated the trigger dependence of the plateau and suggested that entering the second reaction phase was dependent on trigger concentration. As with traditional EXPAR, the limit of detection for the DNA trigger was determined by the nonspecific amplification rates. The optimized traditional template was kinetically distinct from the negative control at 100 fM of initial trigger, and the looped templates were kinetically distinct from the negative control at approximately 1 pM of initial trigger. Nearly all looped templates tested could distinguish between 0 and 10 pM initial trigger concentrations (data not shown).

Analyzing First Phase Kinetics by Weakening Loop Thermodynamics within Templates:

We expect that weakening the loop structure would accelerate reaction kinetics in the first phase; a weaker loop will open and amplify faster than a strong loop. To examine this phenomenon, the free energy of the looped template structure was altered by adding long random sequences of 4-8 nucleotides into the loop before the nickase recognition site. This modification held the palindrome, toehold, and trigger sequences constant while weakening the template loop structure. The only additional thermodynamic parameter this modified was an increase in stability of the long trigger:template complex, which forms after the initial elongation of a template-bound trigger. Long triggers included the original trigger sequence, the nickase recognition site, and the additional long random sequences. The first inflection points of these new long random sequence (lrs) templates were divided by the first inflection point of the base template with no lrs; a relative first inflection point of one denotes a template that was not a□ected by the weakened loop.

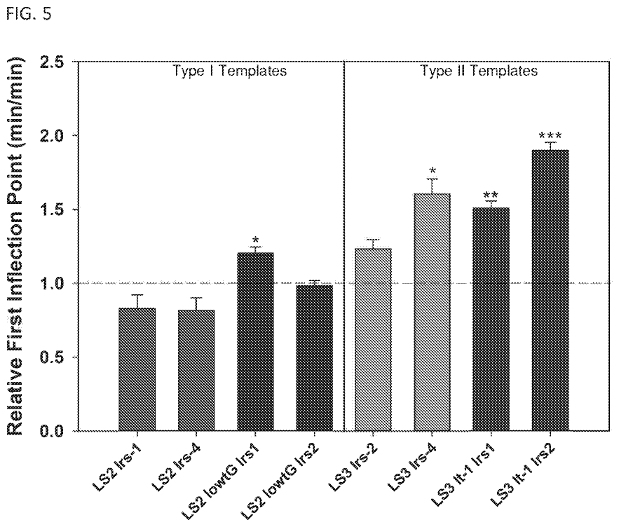

These weakened template loops produced surprising and revealing results. For type I template LS2, decreasing the strength of the loop appeared to cause the loop to open faster, although the p values from a Holm-Bonferroni t-test were not significant (p<0.09 and p<0.06, without correction). Type I template LS2 lowtG had similar or slightly slower trigger production in the first phase when the loop was weakened (FIG. 5). Surprisingly, decreasing the strength of the type II template loops slowed the first reaction phase for every template tested, with significant reaction delay for LS3 It-1 templates and LS3 lrs-4 (FIG. 5). We hypothesize that the increased stability of the long triggers caused this phenomenon; these long triggers were more stable and therefore more di□cult to remove. This observed decrease in the first phase reaction rate supported the hypothesis that templates were deactivated by unextendable “poisoned” complementary strands, and that type II templates were more susceptible to this phenomenon than type I templates.

### Analyzing Acceleration in the Second Reaction Phase:

Rapid acceleration in the second phase would be beneficial if these reactions are used as a digital readout, because a large jump in the second phase resembles definitive switch turn-on. We further analyzed DNA amplification kinetics for their ability to accelerate in the second phase, and to determine if relative second phase kinetics correlated with DNA association thermodynamics. The second phase acceleration was defined as the ratio of the maximum reaction rate in the second phase to the maximum reaction rate in the first phase. We hypothesized that cooperative binding of the trigger to the two toehold binding sites could possibly cause a rapid increase in reaction kinetics in the second phase. Hill coe□cients of a homotropic cooperative receptor increase with the ratio between dissociation constants of the first and second binding events; a large di□erence in stability between the first and second ligand associations will result in a larger Hill coe□cient and greater Hill behaviour. This is qualitatively intuitive: the more relative stability that the first association provides, the greater the kinetic benefit of having a higher ligand concentration. In our system, this corresponds to the amplification incompetent state (a closed template) moving to an amplification competent state (an open template) through dual trigger binding. Free energy is directly proportionally to the natural log of the dissociation constant, so we tested the hypothesis of trigger-mediated co-operative looped template opening using DNA association thermodynamics. Δ G5′ toehold is the free energy of the toehold binding on the 5′ end of the template, Δ G3′ toehold is the free energy of toehold binding on the 3′ end of the template, ΔGpalindrome is the free energy of palindrome association, ΔGloop is the free energy of the template looped secondary structure, and ΔGtrigger:template is the free energy of trigger association with an open template. We characterized the relative dissociation of the first and second trigger binding events by the di⋅erence in the free energy of the first binding event ΔG5′ toehold+ΔG3′ toehold+ΔGpalindrome−ΔGloop and the second binding event ΔGtrigger:template. A larger value signified a greater di⋅erence between the stability of the first and second trigger associations to the template.

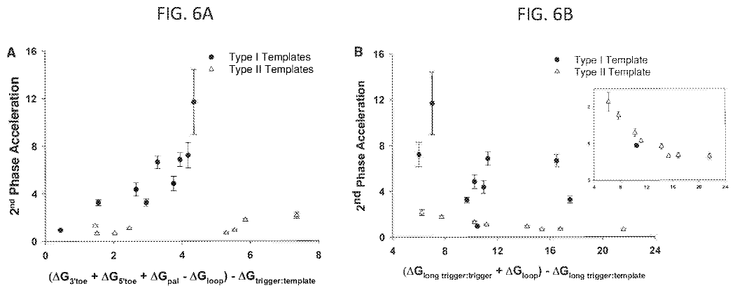

The two template types showed distinct behavior. Type I templates showed significant correlation between the di⋅erence between the first and second trigger binding event and the reaction acceleration in the second phase (Spearman's Rho=0.9667, p<1.7×10⁻⁴), while Type II templates did not (Spearman's Rho=0.6437, p<0.10) (FIG. 6A). The loop melting temperature of Type I templates was higher than the melting temperature of the trigger:template complex (Table 2), but upon association the trigger will open the loop structure and switch the receptor to a binding competent state (FIG. 2A, subpanel 1b).

Type II templates appear to have di□erent dominant reaction pathways. Long triggers are triggers with an elongated nicking endonuclease recognition site. Long trigger removal was hypothesized to be driven by loop closure, which could be hindered by the presence of a stable triggers that remained on type II templates after nicking. The low acceleration in the second phase seen in type II templates could possibly be due to hindered removal of long poisoned triggers, as shown in FIG. 5. FIG. 6B supports this hypothesis. ΔGlong trigger:trigger is the free energy of the long trigger association with trigger, and ΔGlong trigger:template is the free energy of the long trigger association with the template. The parameter: ΔGlong trigger:trigger+ΔGloop−ΔGlong trigger:template approximates thermodynamics of long trigger removal through trigger association to the long trigger and subsequent loop closure, as shown in FIG. 2A (subpanel 4). A larger value corresponds to greater stability of the long triggers, which would slow amplification due to the poor removal of long poisoned triggers. Type II templates significantly correlate with this thermodynamic parameter, with the stability of the long triggers inversely proportional to the reaction acceleration in the second phase (Spearman's Rho=−0.9762, p<4.0×10⁻⁴). This correlation was not significant when analyzing type I templates (Spearman's Rho=−0.3333, p<0.39). These observations support the concept of two distinct templates types, with Type II template kinetics hindered by poor long trigger removal.

### Conclusions

We report a novel, biphasic DNA amplification reaction with a low gain first phase followed by a high gain second phase. The first phase resembles the popular oligonucleotide amplification reaction EXPAR, which operates on similar principles but uses a linear DNA template without a palindromic sequence. We hypothesize that the accumulation of “poisoned” templates with bound unextendable or uncleavable long triggers may slow the reaction and cause the first plateau seen in most palindromic templates and all EXPAR templates. The presence of a palindrome in the template appears to rescue the reaction from this plateau, even when the loop structure is not stable at the reaction temperature (data not shown). While palindromes can rescue the reaction from the first plateau, not all palindromic templates showed this first plateau (data not shown). Several highly stable loops had slow kinetics and unmeasurable plateaus, and it was unclear if they entered the second phase. Two templates with vanishingly small plateaus that entered the second phase both had relatively stable trigger:template complexes, although with only two templates it is not clear from this data the exact parameters that cause the plateau phase to e□ectively disappear.

The reactions investigated here fall into two distinct categories: type I templates have triggers that will dynamically dissociate from the template after nicking (trigger Tm<reaction temperature+5° C., so Tm<60° C. at a reaction temperature of 55° C.), while type II templates had stable trigger template associations that were more likely to remain until strand displacement by the polymerase (trigger Tm>reaction temperature+5° C., so Tm>60° C. at a reaction temperature of 55° C.). DNA association thermodynamics related to the first phase reaction kinetics and second phase acceleration within the two template types, but did not show the same correlations between template types. Long template-bound triggers appeared to slow the reaction, particularly for type II templates which had stable triggers bound after nicking. We hypothesize that these e□ects caused a smaller acceleration in the second phase for type II templates, which did not show the same switch-like jump in second phase product concentration when compared to type I templates. These observations suggested that the two types of templates had di□erent dominant reaction pathways and provided important design considerations to tune the reaction output during each phase. For instance, in order to achieve a high acceleration in the second phase, a type I template with a large di□erence in free energy between the first and second trigger binding events (e.g., ≥1 Kcal/mole) could be designed (FIG. 6A-B). For a more rapid response in the first phase, a type I template could be created such that ΔG3′ toe<ΔGloop.

We have demonstrated a novel new oligonucleotide amplification reaction with a two-stage output that is dependent on the released oligonucleotide trigger molecule. This biphasic DNA amplification reaction is a simple, one-step isothermal amplification reaction; reactions of this type have gained popularity as they do not require temperature cycling and therefore require less energy, hardware, and time. We have described a thermodynamically-based reaction design framework to approximate first phase output, as well as to tune the reaction acceleration in the switch-like second reaction phase. The reaction can report on a variety of analytes: specific proteins, genomic bacterial DNA, viral DNA, microRNA, or mRNA can continuously create input trigger oligonucleotides, making the biphasic DNA amplification reaction broadly applicable to a variety of target molecules. When combined with single molecule amplification, this technique has the potential to be quantitative through digital amplification and detection. The biphasic nature of this reaction makes it well suited for recognition of low-concentration molecules in biological samples, DNA logic gates, and other molecular recognition systems.

### Example 2

### miRNA Transduction and Amplification: miRNA can Trigger the Biphasic Amplification Chemistry

The amplification reaction mixture contained 1× ThermoPol I Buffer [20 mM Tris-HCl (pH 8.8), 10 mM (NH4)2SO4, 10 mM KCl, 2 mM MgSO4, 0.1% Triton® X-100], 25 mM Tris-HCl (pH 8), 6 mM MgSO4, 50 mM KCl, 0.5 mM each dNTP, 0.1 mg/mL BSA, 0.2 U/μL Nt.BstNBI, and 0.0267 U/μL Bst 2.0 WarmStart® DNA Polymerase. Bst 2.0 WarmStart® DNA polymerase is inactive below 45° C.; this decreases non-specific amplification before reaction initiation and theoretically increases experimental reproducibility. Templates were diluted in nuclease-free water and added at a final concentration of 50 nM each. SYBR Green II (10,000× stock in DMSO) was added to the reaction mixture to a final concentration of 5×. The amplification reaction was triggered by the indicated concentration of synthetic target miRNA. 1 ng/μL of MS2 carrier RNA (Roche) was included in all reactions. When indicated 5 μg/mL of thermostable single stranded binding protein (ET-SSB) was added to the reaction. A plus sign (+) to the left of a nucleotide sequence indicates an LNA base. Bold nucleotides indicate the palindrome, italics indicate the toehold region, and underlined nucleotides indicate the nickase recognition site.

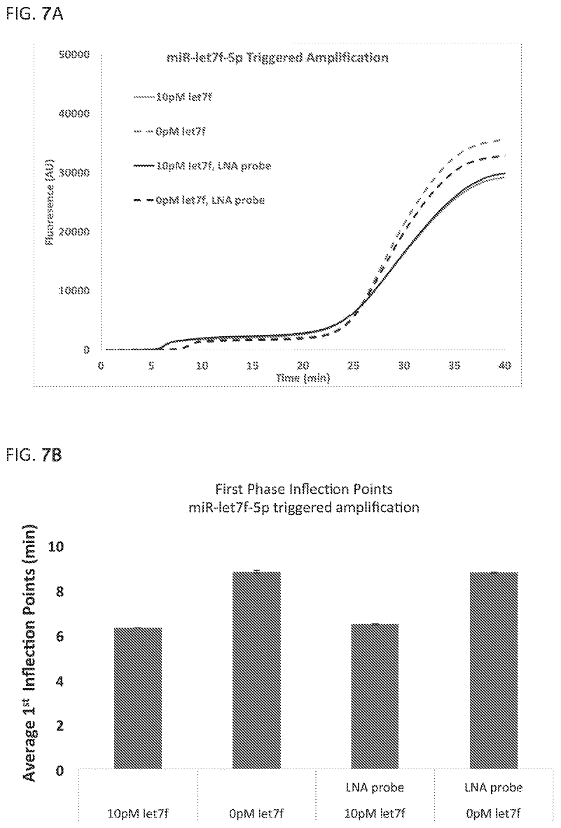

In a first example, the mature miRNA miR-let7f-5p (5′-UGAGGUAGUAGUUGUAUAGUU-3′, SEQ ID NO: 62) was transduced to trigger 5′-CCAAACTCCGGA-3′ (SEQ ID NO: 50, Table 1) in the reaction mixture by using either Transduction template LS31pG3let7f5pLNA (5′-TCCGGAGTTTGGTAATGACTCTAACTA+TACAATC+TACTACC+TC-3′ (PO₃) (SEQ ID NO: 63) or Transduction template LS3lowpG3let7f5p (5′-TCCGGAGTTTGGTAATGACTCTAACTATACAATCTACTACCTCA-3′ NH₂) (SEQ ID NO: 64), and further used in combination with the DNA template LS3 lowpG3 (Table 1). This triggered the biphasic reaction chemistry demonstrating a non-linear amplification rate (e.g., cooperative Hill kinetics). The amplification traces and graphs of corresponding inflection points for these reactions are shown in FIG. 7A and FIG. 7B, respectively. First phase amplification kinetics were distinct at 10 pM of miRNA trigger, which would allow this system to be used in a sink-based switch. The presence of LNA in the transduction template did not significantly affect amplification kinetics. These reactions included single stranded binding protein.

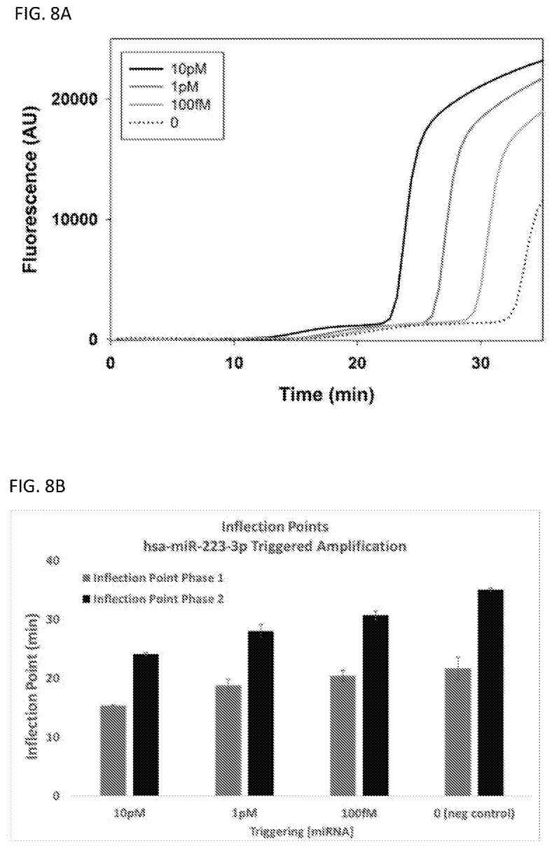

In a second example, the mature miRNA hsa-miR-223-3p (5′-UGUCAGUUUGUCAAAUACCCCA-3′, SEQ ID NO 65) was transduced to the trigger 5′-ATTCTCCGGA-3′ (SEQ ID NO: 37, Table 1) in the reaction mixture by using Transduction template LS2miR223 (5′-TCCGGAGAATTAATGACTCTTCCCCTATTTCACAAACTCACA-3′ NH₂) (SEQ ID NO: 66), and further used in combination with the DNA template LS2 (Table 1). This triggered the biphasic reaction chemistry demonstrating a non-linear amplification rate (e.g., cooperative Hill kinetics). The amplification traces and graphs of corresponding inflection points for these reactions are shown in FIG. 8A and FIG. 8B, respectively. The reaction was kinetically distinct at 100 fM of initial miR-223 concentration.

Various modifications of the present invention, in addition to those shown and described herein, will be apparent to those skilled in the art of the above description. Such modifications are also intended to fall within the scope of the appended claims.

It is appreciated that all reagents are obtainable from commercial sources known in the art unless otherwise specified.

Patents, publications, and applications mentioned in the specification are indicative of the levels of those skilled in the art to which the invention pertains. These patents, publications, and applications are incorporated herein by reference to the same extent as if each individual patent, publication, or application was specifically and individually incorporated herein by reference.

The foregoing description is illustrative of particular aspects of the invention, but is not meant to be a limitation upon the practice thereof.

1. A method of detecting a target oligonucleotide sequence (X), said method comprising:
(A) forming a reaction mixture that comprises:
(1) a target nucleic acid comprising a target oligonucleotide sequence (X);
(2) a first antisense template (X′R1t′YP) that comprises from 3′ to 5′:
(a) a first sequence of nucleotides (X′) that is at least substantially complementary to the target oligonucleotide sequence (X);
(b) a second sequence of nucleotides (R1) that is an anti-sense strand of a first nicking enzyme binding site;
(c) a third sequence of nucleotides (t′Yp) that is at least substantially complementary to a reporter oligonucleotide sequence (tYp), comprising from 3′ to 5′:
(i) a toehold nucleotide sequence (t′); and
(ii) a palindromic nucleotide sequence (Yp);
(3) a second antisense template (t′YpR2t′Yp) that comprises from 3′ to 5′:
(a) a fourth sequence of nucleotides t′Yp;
(b) a fifth sequence of nucleotides (R2) that is an anti-sense strand of a second nicking enzyme binding site;
(c) a sixth sequence of nucleotides t′Yp;
wherein the two palindromic nucleotide sequences (Yp) cause the second antisense template (t′YpR2t′YP) to form a palindrome and fold into a stem and loop configuration;
(4) a polymerase;
(5) a first nicking enzyme that nicks at the first nicking enzyme binding site;
(6) a second nicking enzyme that nicks at the second nicking enzyme binding site;
(7) nucleotides;
(B) subjecting the reaction mixture to essentially isothermal conditions at a reaction temperature to amplify the reporter oligonucleotide sequence (tYp) at a non-linear amplification rate; and
(C) detecting the amplified reporter oligonucleotide sequence (tYp).

2. The method of claim 1, wherein the reporter oligonucleotide sequence (tYp) is linearly amplified from the steps comprising:
(A) forming a duplex (D1) comprising the target oligonucleotide sequence (X) and the first antisense template (X′R1t′ YP);
(B) extending, using the polymerase, the target oligonucleotide sequence (X) of the duplex (D1) along the first antisense template (X′R1t′YP) to form an extended target oligonucleotide sequence comprising a sense sequence complementary to the first antisense template (X′R1t′ YP);
(C) nicking, with the first nicking enzyme, at the first nicking enzyme binding site on the sense strand of the duplex (D1) to produce the reporter oligonucleotide sequence (tYp); and
(D) repeating steps (B) and (C) to thereby linearly amplify the reporter oligonucleotide sequence (tYp).

3. The method of any one of claim 1, wherein the reporter oligonucleotide (tYp) is amplified at a non-linear amplification rate from the steps comprising:
(A) forming a duplex (D2) comprising the reporter oligonucleotide sequence (tYp) and the second antisense template (t′YpR2t′ Yp), wherein binding of the reporter oligonucleotide sequence (tYp) to the toehold site (t′) unfolds the stem and loop configuration of the second antisense template (t′YpR2t′Yp);
(B) extending, using the polymerase, the reporter oligonucleotide sequence (tYp) of the duplex (D2) along the second antisense template (t′YpR2t′Yp) to form an extended reporter oligonucleotide sequence comprising a sense sequence complementary to the second antisense template (t′YpR2t′Yp);
(C) nicking, with the second nicking enzyme, at the second nicking enzyme binding site on the sense strand of the duplex (D2) to produce the reporter oligonucleotide sequence (tYp); and
(D) repeating steps (B) and (C) to thereby amplify the reporter oligonucleotide sequence (tYp) at a non-linear amplification rate.

4. The method of claim 1, wherein the non-linear amplification rate of the reporter oligonucleotide sequence (tYp) demonstrates cooperative Hill kinetics.

5. The method of claim 3, wherein the non-linear amplification rate of the reporter oligonucleotide sequence (tYp) demonstrates cooperative Hill kinetics.

6. The method of claim 1, wherein the amplification of the reporter oligonucleotide sequence (tYp) is biphasic, and wherein the first phase linearly amplifies the oligonucleotide sequence (tYp) and the second phase amplifies the reporter oligonucleotide sequence (tYp) at a non-linear amplification rate.

7. The method of claim 1, wherein said method can detect the target oligonucleotide sequence (X) at a concentration of ≤10 picomolar.

8. The method of claim 1, wherein first nicking enzyme binding site is identical to the second nicking enzyme binding site.

9. The method of claim 1, wherein first nicking enzyme binding site is identical to the second nicking enzyme binding site are nicked by the same nicking enzyme.

10. The method of claim 1, wherein the first sequence of nucleotides (X′) is completely complementary to the target oligonucleotide sequence (X).

11. The method of claim 1, wherein the third sequence of nucleotides (t′Yp) is completely complementary to the reporter oligonucleotide sequence (tYp).

12. The method of claim 1, wherein the third sequence of nucleotides (t′Yp) is non-complementary to the target oligonucleotide sequence (X).

13. The method of claim 1, wherein the 3′ terminus of the first antisense template (X′R1t′YP) and the 3′ terminus of the second antisense template (t′YpR2t′YP) are blocked.

14. The method of claim 1, wherein the step of detecting the amplified reporter oligonucleotide sequence (tYp) is performed at least partially by luminescence spectroscopy or spectrometry, fluorescence, fluorescence spectroscopy or spectrometry, mass spectrometry, liquid chromatography, fluorescence polarization, colorimetry, electrophoresis, or a combination thereof.

15. The method of claim 1, wherein detecting reporter oligonucleotide sequence (tYp) comprises detecting an amplification rate of the reporter oligonucleotide sequence (tYp).

16. The method of claim 15, wherein the step of detecting the amplification rate of the reporter oligonucleotide sequence (tYp) is performed at least partially by luminescence spectroscopy or spectrometry, fluorescence, fluorescence spectroscopy or spectrometry, mass spectrometry, liquid chromatography, fluorescence polarization, colorimetry, electrophoresis, or a combination thereof.

17. The method of claim 1, wherein the target nucleic acid comprising a target oligonucleotide sequence (X) is obtained from a sample derived from an animal.

18. The method of claim 17, wherein the sample is blood, serum, mucus, saliva, urine, or feces.

19. The method of claim 1, wherein the target nucleic acid comprising a target oligonucleotide sequence (X) is any synthetic or natural RNA molecule, including mRNA, microRNA, and siRNA.

20. The method of claim 1, wherein the target nucleic acid comprising a target oligonucleotide sequence (X) is any synthetic or natural DNA molecule, including genomic DNA, mitochondrial DNA, cDNA derived from reverse transcription of mRNA, microRNA, or siRNA, and wherein said method comprises a step of denaturing or cleaving said target nucleic acid comprising a target oligonucleotide sequence (X) prior to forming the reaction mixture.

21. The method of claim 1, wherein said polymerase is a warm start polymerase.

22. The method of claim 1, wherein amplification of the reporter oligonucleotide sequence (tYp) is performed at about 54° C. to about 60° C.

23. The method of claim 1, wherein said reporter oligonucleotide sequence (tYp) is from 8-30 nucleotides in length.

24. The method of claim 1, wherein the toehold site (t′) of the first, third, fourth, and fifth sequence of nucleotides is from 3-8 nucleotides in length.

25. The method of claim 1, wherein the palindrome of the second antisense template (t′YpR2t′YP) is from 4-22 nucleotides in length.

26. The method of claim 1, wherein the palindrome of the second antisense template (t′YpR2t′ YP) has a melting temperature that is greater than the reaction temperature, but less than 90° C.

27. The method of claim 3, wherein the duplex (D2) has a melting temperature that is less than the reaction temperature plus 5° C.

28. The method of claim 9, wherein the nicking enzyme is selected from the group consisting of Nt.BstNBI, Nt.BspQI, Nb.BBvCI, Nb.BsmI, Nb.BsrDI, Nb.Bstl, Nt.Alwl, Nt.BbvCI, Nt.CviPII, Nt.BsmAI, Nb.Bpu1oI, and Nt.Bpu10I.


SEQUENCE LISTING (66 sequences)

SEQ ID NO: 1 - DNA, 30 bp, Artificial Sequence
  template LS2
tccggagaattaatgactcttccggagaat

SEQ ID NO: 2 - DNA, 34 bp, Artificial Sequence
  template LS3
cgcgcggtttggtaatgactctcgcgcggtttgg

SEQ ID NO: 3 - DNA, 30 bp, Artificial Sequence
  template LS2-1
accggtgaattaatgactctaccggtgaat

SEQ ID NO: 4 - DNA, 30 bp, Artificial Sequence
  template LS2-2
agcgctgaattaatgactctagcgctgaat

SEQ ID NO: 5 - DNA, 30 bp, Artificial Sequence
  template LS2-3
tgcgcagaattaatgactcttgcgcagaat

SEQ ID NO: 6 - DNA, 30 bp, Artificial Sequence
  template LS2-4
tccggagaataaaagactcttccggagaat

SEQ ID NO: 7 - DNA, 30 bp, Artificial Sequence
  tempate LS2-5
tccggagaataaaggactcttccggagaat

SEQ ID NO: 8 - DNA, 30 bp, Artificial Sequence
  template LS2-6
tggccagaattaatgactcttggccagaat

SEQ ID NO: 9 - DNA, 30 bp, Artificial Sequence
  template LS2-7
tcgcgagaattaatgactcttcgcgagaat

SEQ ID NO: 10 - DNA, 30 bp, Artificial Sequence
  template LS2-8
tccggacaattaatgactcttccggacaat

SEQ ID NO: 11 - DNA, 34 bp, Artificial Sequence
  template LS2lrs-1
tccggagaattaatgactctgctttccggagaat

SEQ ID NO: 12 - DNA, 39 bp, Artificial Sequence
  LS2lrs-4
ccggagaattaatgactctgcttagtcagtccggagaat

SEQ ID NO: 13 - DNA, 40 bp, Artificial Sequence
  template LS3lrs-2
cgcgcggtttggtaatgactctgcttagcgcgcggtttgg

SEQ ID NO: 14 - DNA, 42 bp, Artificial Sequence
  template LS3lrs-4
cgcgcgtttggtaatgactctacttggcttacgcgcggttgg

SEQ ID NO: 15 - DNA, 34 bp, Artificial Sequence
  template LS52lowtG lrs1
tccggataattaatgactctgctttccggataat

SEQ ID NO: 16 - DNA, 35 bp, Artificial Sequence
  template LS2lowtglrs2
tccggataattaatgactgctgctttccggataat

SEQ ID NO: 17 - DNA, 44 bp, Artificial Sequence
  template LS3 lt-1lrs1
cgcgcggtttggacgtaatgactctgcttcgcgcggtttggacg

SEQ ID NO: 18 - DNA, 50 bp, Artificial Sequence
  LS3lt-1rs2
cgcgcggtttggacgtaatgactctgcttagtcagcgcgcggtttggacg

SEQ ID NO: 19 - DNA, 35 bp, Artificial Sequence
  LS3lp
cgcgcgcggtttgtaatgactctcgcgcgcggttg

SEQ ID NO: 20 - DNA, 34 bp, Artificial Sequence
  template LS3lowpG3
tccggagtttggtaatgactcttccggagtttgg

SEQ ID NO: 21 - DNA, 30 bp, Artificial Sequence
  template LS3 sp
cgcggtttattaatgactctcgcggtttat

SEQ ID NO: 22 - DNA, 34 bp, Artificial Sequence
  template LS3lowpG2
tagctagtttggtaatgactcttagctagtttgg

SEQ ID NO: 23 - DNA, 30 bp, Artificial Sequence
  template LS2hpG1
gccggcgaattaatgactctgccggcgaat

SEQ ID NO: 24 - DNA, 30 bp, Artificial Sequence; identical to SEQ ID NO: 23
  template LS2lp

SEQ ID NO: 25 - DNA, 26 bp, Artificial Sequence
  template LS2 sp
ccgggaattaatgactctccgggaat

SEQ ID NO: 26 - DNA, 40 bp, Artificial Sequence
  template LS3 lt1
cgcgcggtttggacgtaatgactctcgcgcggtttggacg

SEQ ID NO: 27 - DNA, 34 bp, Artificial Sequence
  template LS3 htG
cgcgcggtgcggtaatgactctcgcgcggtgcgg

SEQ ID NO: 28 - DNA, 34 bp, Artificial Sequence
  template LS3 lowtG
cgcgcggtttattaatgactctcgcgcggtttat

SEQ ID NO: 29 - DNA, 30 bp, Artificial Sequence
  template LS2 htG2
tccggagcgctaatgactcttccggagcgc

SEQ ID NO: 30 - DNA, 38 bp, Artificial Sequence
  template LS2 lt3
tccggagaatgatctaatgactcttccggagaatgatc

SEQ ID NO: 31 - DNA, 26 bp, Artificial Sequence
  template LS2 s
tccggagataatgactcttccggaga

SEQ ID NO: 32 - DNA, 30 bp, Artificial Sequence
  template LS2 lowtG
tccggataattaatgactcttccggataat

SEQ ID NO: 33 - DNA, 34 bp, Artificial Sequence
  LS2no5
tccggagaattaatgactctattctccggagaat

SEQ ID NO: 34 - DNA, 42 bp, Artificial Sequence
  template LS2 no5′lrs3
tccggagaattaatgactctgtatagctattctccggagaat

SEQ ID NO: 35 - DNA, 30 bp, Artificial Sequence
  Ran2
ggggaaataggtgagactctggggaaatag

SEQ ID NO: 36 - DNA, 30 bp, Artificial Sequence
  template EXPAR1
ctcacgctacggacgactctctcacgctac

SEQ ID NO: 37 - DNA, 10 bp, Artificial Sequence
  trigger
attctccgga

SEQ ID NO: 38 - DNA, 11 bp, Artificial Sequence
  trigger
caaaccgcgcg

SEQ ID NO: 39 - DNA, 10 bp, Artificial Sequence
  trigger
attcaccggt

SEQ ID NO: 40 - DNA, 10 bp, Artificial Sequence
  trigger
attcagcgct

SEQ ID NO: 41 - DNA, 10 bp, Artificial Sequence
  trigger
attctgcgca

SEQ ID NO: 42 - DNA, 10 bp, Artificial Sequence
  trigger
attctggcca

SEQ ID NO: 43 - DNA, 10 bp, Artificial Sequence
  trigger
attctcgcga

SEQ ID NO: 44 - DNA, 10 bp, Artificial Sequence
  trigger
attgtccgga

SEQ ID NO: 45 - DNA, 12 bp, Artificial Sequence
  trigger
ccaaaccgcgcg

SEQ ID NO: 46 - DNA, 10 bp, Artificial Sequence
  trigger
attatccgga

SEQ ID NO: 47 - DNA, 12 bp, Artificial Sequence
  trigger
ccaaactagcta

SEQ ID NO: 48 - DNA, 15 bp, Artificial Sequence
  trigger
cgtccaaaccgcgcg

SEQ ID NO: 49 - DNA, 13 bp, Artificial Sequence
  trigger
caaaccgcgcgcg

SEQ ID NO: 50 - DNA, 12 bp, Artificial Sequence
  trigger
ccaaactccgga

SEQ ID NO: 51 - DNA, 10 bp, Artificial Sequence
  trigger
ccaaaccgcg

SEQ ID NO: 52 - DNA, 10 bp, Artificial Sequence
  trigger
attcgccggc

SEQ ID NO: 53 - DNA, 8 bp, Artificial Sequence
  trigger
attcccgg

SEQ ID NO: 54 - DNA, 12 bp, Artificial Sequence
  trigger
ccgcaccgcgcg

SEQ ID NO: 55 - DNA, 12 bp, Artificial Sequence
  trigger
ataaaccgcgcg

SEQ ID NO: 56 - DNA, 10 bp, Artificial Sequence
  trigger
gcgctccgga

SEQ ID NO: 57 - DNA, 14 bp, Artificial Sequence
  trigger
gatcattctccgga

SEQ ID NO: 58 - DNA, 8 bp, Artificial Sequence
  trigger
tctccgga

SEQ ID NO: 59 - DNA, 10 bp, Artificial Sequence
  trigger
attatccgga

SEQ ID NO: 60 - DNA, 10 bp, Artificial Sequence
  trigger
ctatttcccc

SEQ ID NO: 61 - DNA, 10 bp, Artificial Sequence
  trigger
gtagcgtgag

SEQ ID NO: 62 - RNA, 21 nt, Artificial Sequence
  miRNA miR-let7f-5p
ugagguaguaguuguauaguu

SEQ ID NO: 63 - DNA, 43 bp, Artificial Sequence
  template LS3lpG3let7f5pLNA
tccggagtttggtaatgactctaactatacaatctactacctc

SEQ ID NO: 64 - DNA, 44 bp, Artificial Sequence
  template LS3lowpG3let7f5p
tccggagtttggtaatgactctaactatacaatctactacctca

SEQ ID NO: 65 - RNA, 22 nt, Homo sapiens
ugucaguuugucaaauacccca

SEQ ID NO: 66 - DNA, 42 bp, Artificial Sequence
  template LS2miR223
tccggagaattaatgactcttcccctatttcacaaactcaca